(19) United States
(12) Patent Application Publication
(10) Pub. No.: US 2024/0382068 A1
(43) Pub. Date: Nov. 21, 2024

(54) IMAGE PROCESSING DEVICE, ENERGY TREATMENT INSTRUMENT, TREATMENT SYSTEM, AND IMAGE PROCESSING METHOD

(71) Applicant: Olympus Corporation, Tokyo (JP)

(72) Inventors: Hiroshi SUZUKI, Tokyo (JP); Koichiro WATANABE, Tokyo (JP)

(21) Appl. No.: 18/785,400

(22) Filed: Jul. 26, 2024

Related U.S. Application Data
(63) Continuation of application No. PCT/JP2022/010712, filed on Mar. 10, 2022.

(51) Int. Cl. A61B 1/00; A61B 18/00; A61B 34/20; A61B 90/00; G06T 5/50; G06T 5/92; G06T 7/00

(52) U.S. Cl. CPC A61B 1/000095; A61B 1/00045; A61B 18/00; A61B 34/20; G06T 5/50; G06T 5/92; G06T 7/0012; A61B 2018/00982; A61B 2034/2065; A61B 2090/3937; G06T 2207/10068; G06T 2207/20192; G06T 2207/20212; G06T 2207/30168

(57) ABSTRACT

An image processing device includes a processor including hardware, the processor being configured to: acquire image data partially including a first region in which a living body is treated with at least an energy treatment instrument; detect a second region including at least a part of the energy treatment instrument from an image corresponding to the image data; generate enhancement image data in which the second region is enhanced compared to regions other than the second region, based on the image data and a detection result of the second region; perform tone correction on the image corresponding to the image data to generate correction image data; and generate first composite image data in which the correction image data and the enhancement image data are combined.

### CROSS-REFERENCE TO RELATED APPLICATION

This application is a continuation of International Application No. PCT/JP2022/010712, filed on Mar. 10, 2022, the entire contents of which are incorporated herein by reference.

### BACKGROUND

### 1. Technical Field

The present disclosure relates to an image processing device, an energy treatment instrument, a treatment system, and an image processing method.

### 2. Related Art

In arthroscopic surgery, a technique that uses an irrigation device to inflate the inside of a joint with irrigation fluid, such as physiological saline solution, to secure a field of view and perform a procedure on a treatment site has been known (for example, Japanese Patent No. 4564595). In this technique, because bone powder, which is scrapings of bone, and marrow fluid are generated by crushing a bone with a hammering action of an ultrasound treatment instrument, the visibility of a treatment site is ensured by expelling bone powder and marrow fluid from the field of view of an endoscope with irrigation fluid.

### SUMMARY

In some embodiments, an image processing device includes a processor including hardware, the processor being configured to: acquire image data partially including a first region in which a living body is treated with at least an energy treatment instrument; detect a second region including at least a part of the energy treatment instrument from an image corresponding to the image data; generate enhancement image data in which the second region is enhanced compared to regions other than the second region, based on the image data and a detection result of the second region; perform tone correction on the image corresponding to the image data to generate correction image data; and generate first composite image data in which the correction image data and the enhancement image data are combined.

In some embodiments, provided is an energy treatment instrument that can be inserted into a subject and is capable of treating a treatment target site. The energy treatment instrument is configured to be captured by an endoscope inserted into the subject. The energy treatment instrument includes an indicator that is arranged at a distal end of the energy treatment instrument, the indicator being configured to be detected by enhancing a region included in a part of the energy treatment instrument in an image corresponding to image data captured by the endoscope compared to other regions in an edge component and a brightness.

In some embodiments, a treatment system includes: an energy treatment instrument that can be inserted into a subject, and that is capable of treating a treatment target site; an endoscope that can be inserted into the subject, and that is capable of generating image data by capturing a first region in which a living body is treated with at least the energy treatment instrument; and an image processing device configured to perform image processing with respect to the image data to output to a display, the image processing device including a processor including hardware, the processor being configured to: acquire the image data; detect a second region including at least a part of the energy treatment instrument from an image corresponding to the image data; generate enhancement image data in which the second region is enhanced compared to regions other than the second region, based on the image data and a detection result of the second region; perform tone correction on the image corresponding to the image data to generate correction image data; and generate composite image data in which the correction image data and the enhancement image data are combined.

In some embodiments, provided is an image processing method that is performed by an image processing device including a processor including hardware, the method including: acquiring image data partially including a first region in which a living body is treated with at least an energy treatment instrument; detecting a second region including at least a part of the energy treatment instrument from an image corresponding to the image data; generating enhancement image data in which the second region is enhanced compared to regions other than the second region, based on the image data and a detection result of the second region; performing tone correction on the image corresponding to the image data to generate correction image data; and generating first composite image data in which the correction image data and the enhancement image data are combined.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of a treatment system according to a first embodiment of the present disclosure;

FIG. 2 is a diagram illustrating a state in which a bone hole is formed by an ultrasound probe according to the first embodiment of the present disclosure;

FIG. 3A is a schematic diagram illustrating a schematic configuration of the ultrasound probe according to the first embodiment of the present disclosure;

FIG. 3B is a schematic diagram illustrating a direction of an arrow A in FIG. 3A;

FIG. 4 is a block diagram illustrating an overview of a functional configuration of the entire treatment system according to the first embodiment of the present disclosure;

FIG. 5 is a block diagram illustrating a detailed functional configuration of an endoscope device according to the first embodiment of the present disclosure;

FIG. 6A is a diagram illustrating a state in which a field of view of the endoscope according to the first embodiment of the present disclosure is in a good condition;

FIG. 6B is a diagram illustrating a state in which a field of view of the endoscope according to the first embodiment of the present disclosure is in a poor condition;

FIG. 7 is a block diagram illustrating a detailed functional configuration of a processing device according to the first embodiment of the present disclosure;

FIG. 8 is a block diagram illustrating a detailed functional configuration of an irrigation device according to the first embodiment of the present disclosure;

FIG. 9 is a block diagram illustrating a detailed functional configuration of an illumination device according to the first embodiment of the present disclosure;

FIG. 10 is a block diagram illustrating a functional configuration of an image processing unit according to the first embodiment of the present disclosure;

FIG. 11 is a block diagram illustrating a detailed functional configuration of a correction-image generating unit according to the first embodiment of the present disclosure;

FIG. 12 is a flowchart explaining an overview of a procedure performed by an operator by using the treatment system according to the first embodiment of the present disclosure;

FIG. 13 is a diagram explaining about an overview of processing performed in a cutting procedure by an endoscope control device according to the first embodiment of the present disclosure;

FIG. 14 is a flowchart illustrating a detailed overview of haze-treatment control processing in FIG. 13:

FIG. 15 is a diagram illustrating an example of a display image in a state in which a field of view of the endoscope according to the first embodiment of the present disclosure is in a good condition;

FIG. 16 is a diagram illustrating a relationship between a position and a brightness on a line A-A′ in FIG. 15;

FIG. 17 is a diagram illustrating an example of a display image in a state in which a field of view of the endoscope according to the first embodiment of the present disclosure is in a poor condition;

FIG. 18 is a diagram illustrating a relationship between a position and a brightness on the line A-A′ in FIG. 16;

FIG. 19 is a diagram illustrating a relationship between a position and a brightness on the line A-A′ after a correction-image generating unit performs tone correction with respect to the display image in FIG. 17;

FIG. 20 is a diagram illustrating a relationship between a position and a brightness of an enhancement image generated by an enhancement-image generating unit according to the first embodiment of the present disclosure;

FIG. 21 is a diagram schematically illustrating a generation method of a composite image generated by a composite-image generating unit according to the first embodiment of the present disclosure;

FIG. 22 is a diagram illustrating an example of a display image displayed by a display device according to the first embodiment of the present disclosure;

FIG. 23 is a block diagram illustrating a functional configuration of an image processing unit according to a second embodiment of the present disclosure;

FIG. 24 is a flowchart illustrating a detailed overview of the haze-treatment control processing performed by an endoscope control device according to the second embodiment of the present disclosure;

FIG. 25 is a diagram schematically illustrating a generation method of a composite image generated by a composite-image generating unit according to the second embodiment of the present disclosure;

FIG. 26 is a block diagram illustrating a functional configuration of an image processing unit according to a third embodiment of the present disclosure;

FIG. 27 is a schematic diagram illustrating a schematic configuration of a part of a treatment instrument according to the third embodiment of the present disclosure;

FIG. 28 is a diagram illustrating an example of a display image in which a part of the treatment instrument according to the third embodiment of the present disclosure is captured;

FIG. 29 is a diagram illustrating a relationship between a position and a brightness on the line A-A′ in FIG. 28;

FIG. 30 is a diagram illustrating a relationship between a position and a brightness on a line same as the line A-A′ in FIG. 28 in a high dynamic range (HDR) image generated by an HDR-image generating unit according to the third embodiment of the present disclosure;

FIG. 31 is a diagram illustrating a relationship between a position and a brightness on a line same as the line A-A′ in FIG. 28 in a display image in a state in which the field of view of an endoscope according to the third embodiment of the present disclosure is in a poor condition;

FIG. 32 is a diagram illustrating a relationship between a position and a brightness on a line same as the line A-A′ in FIG. 28 in a composite image generated by a composite-image generating unit according to the third embodiment of the present disclosure;

FIG. 33 is a block diagram illustrating a functional configuration of an image processing device according to a fourth embodiment of the present disclosure; and

FIG. 34 is a block diagram illustrating a functional configuration of a treatment instrument according to the fourth embodiment of the present disclosure.

### DETAILED DESCRIPTION

Hereinafter, embodiments to implement the present disclosure will be explained in detail with reference to the drawings. The following embodiments are not intended to limit the present disclosure. Moreover, the respective drawings to be referred to in the following explanation only provide a schematic representation of shape, size, and positional relationship to the extent that the content of the present disclosure can be understood. That is, the present disclosure is not limited to the shapes, the sizes, and the positional relationships provided in the respective drawings. Furthermore, in the following explanation, identical reference symbols are assigned to identical parts in description of the drawings.

### First Embodiment

### Schematic Configuration of Processing System

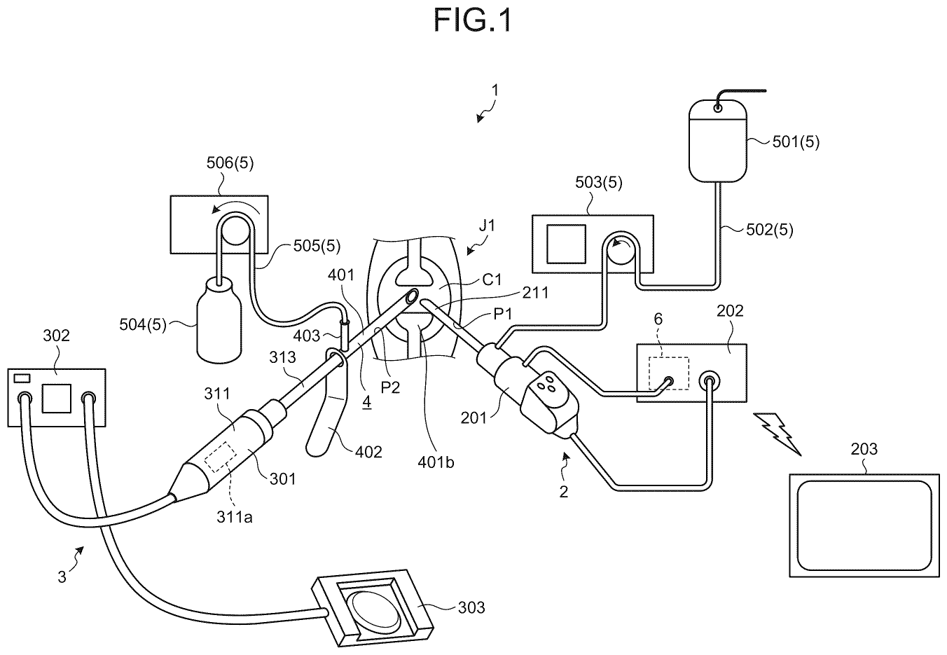

FIG. 1 is a diagram illustrating a schematic configuration of a treatment system 1 according to a first embodiment. The treatment system 1 illustrated in FIG. 1 applies ultrasound vibrations to a living tissue, such as bones, to thereby treat the living tissue. The treatment herein is, for example, removal or excision of a living tissue, such as bones. In FIG. 1, as the treatment system 1, a treatment system that performs anterior cruciate ligament reconstruction surgery is exemplified.

The treatment system 1 illustrated in FIG. 1 includes an endoscope device 2, a treatment device 3, a guiding device 4, an irrigation device 5, and an illumination device 6.

### Configuration of Endoscope Device

First, a configuration of the endoscope device 2 will be explained.

The endoscope device 2 includes an endoscope 201, an endoscope control device 202, and a display device 203.

The endoscope 201 has an insertion portion 211, a distal end portion of which is inserted into a joint cavity C1 through a first portal P1 that communicates an inside of the joint cavity C1 of a knee joint J1 of a subject and an outside of a skin. The endoscope 201 illuminates the inside of the joint cavity C1, captures illumination light (subject image) reflected in the inside of the joint cavity C1, and images a subject image to generate image data.

The endoscope control device 202 performs various kinds of image processing with respect to the image data captured by the endoscope 201, and displays a display image corresponding to the image data subjected to this image processing on the display device 203. The endoscope control device 202 is connected to the endoscope 201 and the display device 203 wiredly or wirelessly.

The display device 203 receives data transmitted from respective devices constituting the treatment system 1, image data (display image), sound data, and the like through the endoscope control device 202, and performs display of the display image, notification, and output according to the received data. The display device 203 is constituted of a display panel of liquid crystal or organic electro-luminescence (EL).

Configuration of Processing device

Next, a configuration of the treatment device 3 will be explained.

The treatment device 3 includes a treatment instrument 301, a treatment-instrument control device 302, and a foot switch 303.

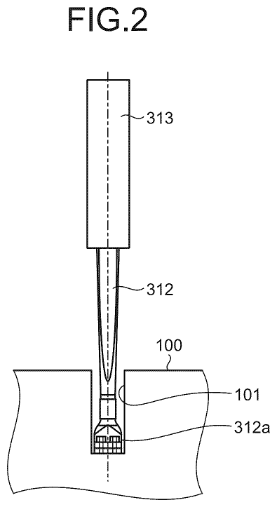

The treatment instrument 301 includes a treatment-instrument main unit 311, an ultrasound probe 312 (refer to FIG. 2 described later), and a sheath 313.

The treatment-instrument main unit 311 is formed in a cylindrical shape. Moreover, inside the treatment-instrument main unit 311, an ultrasound transducer 312a (refer to FIG. 2 described later) that is constituted of a bolt-clamped Langevin-type transducer, and that generates ultrasound vibrations according to a supplied driving power is housed.

The treatment-instrument control device 302 supplies a driving power to the ultrasound transducer 312a according to an operation to the foot switch 303 by an operator. Supply of the driving power is not limited to be performed by operation of the foot switch 303, but may also be performed, for example, according to an operation of an operating unit (not illustrated) provided in the treatment instrument 301.

The foot switch 303 is an input interface for the operator to operate with foot when the ultrasound probe 312 is to be activated.

Next, the ultrasound probe 312 will be explained.

FIG. 2 is a diagram illustrating a state in which a bone hole 101 is formed by the ultrasound probe 312.

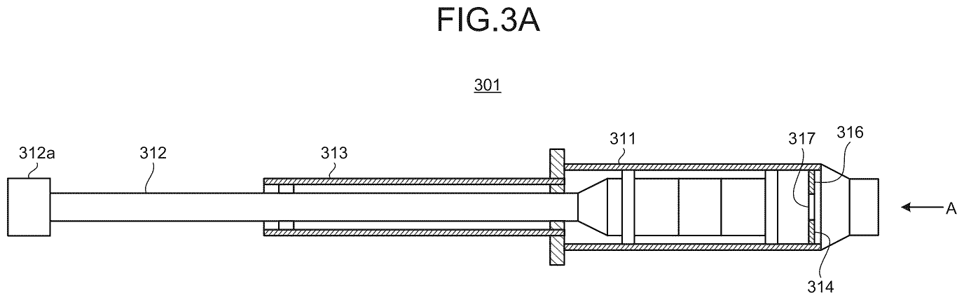

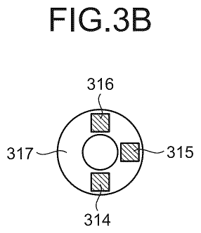

FIG. 3A is a schematic diagram illustrating a schematic configuration of the ultrasound probe 312.

FIG. 3B is a schematic diagram illustrating a direction of an arrow A in FIG. 3A.

In the first embodiment, the ultrasound probe 312 functions as an energy treatment instrument.

As illustrated in FIG. 2, FIG. 3A, and FIG. 3B, the ultrasound probe 312 is constituted of, for example, titanium alloy or the like, and has a substantially cylindrical shape. Moreover, a proximal end portion of the ultrasound probe 312 is connected to the ultrasound transducer 312a inside the treatment-instrument main unit 311. Furthermore, the ultrasound probe 312 transmits ultrasound vibrations generated by the ultrasound transducer 312a from a proximal end to a distal end. Specifically, ultrasound vibrations in the first embodiment are vertical vibrations along a longitudinal direction of the ultrasound probe 312 (up and down direction in FIG. 2). Moreover, at a distal end portion of the ultrasound probe 312, the ultrasound transducer 312a is provided as illustrated in FIG. 2.

The sheath 313 is formed in a cylindrical shape thinner and longer than the treatment-instrument main unit 311, and covers an outer circumference of the ultrasound probe 312 up to an arbitrary length from the treatment-instrument main unit 311.

The ultrasound transducer 312a of the ultrasound probe 312 in the treatment instrument 301 thus configured is inserted into the joint cavity C1 while being guided by the guiding device 4 inserted into the joint cavity C1 through a second portal P2 that communicates between the inside of the joint cavity C1 and an outside of the skin.

Subsequently, the treatment instrument 301 generates ultrasound vibrations in a state in which the ultrasound transducer 312a of the ultrasound probe 312 is in contact with a treatment target site 100 of a bone. A portion of the bone mechanically impacted with the ultrasound transducer 312a is then crushed into fine particles by hammering action (refer to FIG. 2).

Thereafter, when the ultrasound transducer 312a of the ultrasound probe 312 is pushed against the treatment target site 100 by the operator, the treatment instrument 301 enters into the treatment target site 100 while crashing the bone with the ultrasound transducer 312a. Thus, the bone hole 101 is formed in the treatment target site 100.

Moreover, at a proximal end of the treatment-instrument main unit 311, a circuit board 317 on which a posture detecting unit 314, a central processing unit (CPU) 315, a memory 316 are mounted is arranged (refer to FIG. 3A and FIG. 3B).

The posture detecting unit 314 includes a sensor that detects rotation or movement of the treatment instrument 301. The posture detecting unit 314 detects three axes perpendicular to one another, including an axis parallel to a longitudinal axis of the ultrasound probe 312, and rotation about the respective axes. The treatment-instrument control device 302 described above determines that the treatment instrument 301 is in a still state when detection results of the posture detecting unit 314 do not change for a predetermined time.

The CPU 315 controls operation of the posture detecting unit 314, or transmits and receives information to and from the treatment-instrument control device 302. The CPU 315 loads a program stored in the memory 316 onto a work area of a memory to execute, and controls the respective components and the like through execution of the program by a processor, and hardware and software thereby cooperate to implement a functional module that matches a predetermined purpose.

### Configuration of Guiding Device

Next, a configuration of the guiding device 4 will be explained.

The guiding device 4 is inserted in to the joint cavity C1 through the second portal P2, and guides the distal end portion of the ultrasound probe 312 in the treatment instrument 301 to the inside of the joint cavity C1.

The guiding device 4 includes a guide main unit 401, a handle portion 402, and a cock-equipped drainage unit 403.

The guide main unit 401 has a tubular shape, and has a through hole 401a in which the ultrasound probe 312 is inserted (refer to FIG. 1). The guide main unit 401 controls travel of the ultrasound probe 312 inserted in the through hole 401a to be in a certain direction, and guides the movement of the ultrasound probe 312. In the first embodiment, a cross-sectional shape perpendicular to a center axis on an outer peripheral surface and an inner peripheral surface of the guide main unit 401 is a substantially circular shape. Moreover, the guide main unit 401 is tapered to become thinner toward its distal end. That is, a distal end surface 401b of the guide main unit 401 has an inclined surface that intersects the center axis at an angle.

The cock-equipped drainage unit 403 is arranged on an outer peripheral surface of the guide main unit 401, and has a tubular shape that communicates with an interior of the guide main unit 401. To the cock-equipped drainage unit 403, one end of a drainage tube 505 of the irrigation device 5 is connected, to be a flow channel communicating between the guide main unit 401 and the drainage tube 505 of the irrigation device 5. This flow channel is configured to be openable and closable by operating a cock (not illustrated) arranged in the cock-equipped drainage unit 403.

### Configuration of Irrigation Device

Next, a configuration of the irrigation device 5 will be explained.

The irrigation device 5 feeds irrigation fluid, such as sterilized saline solution, to the inside of the joint cavity C1, and discharges the irrigation fluid to the outside of the joint cavity C1.

The irrigation device 5 includes a liquid source 501, a liquid feeding tube 502, an infusion pump 503, a drainage bottle 504, the drainage tube 505, and a drainage pump 506 (refer to FIG. 1).

The liquid source 501 contains irrigation fluid thereinside. To the liquid source 501, the liquid feeding tube 502 is connected. The irrigation fluid is sterilized saline solution or the like. The liquid source 501 is constituted of, for example, a bottle, or the like.

One end of the liquid feeding tube 502 is connected to the liquid source 501, and the other end thereof is connected to the endoscope 201.

The infusion pump 503 sends out the irrigation fluid toward the endoscope 201 from the liquid source 501 through the liquid feeding tube 502. The irrigation fluid sent to the endoscope 201 is sent to the inside of the joint cavity C1 from a liquid feeding hole formed in a distal end portion of the insertion portion 211.

The drainage bottle 504 contains irrigation fluid that is discharged out from the joint cavity C1. To the drainage bottle 504, the drainage tube 505 is connected.

One end of the drainage tube 505 is connected to the guiding device 4, and the other end thereof is connected to the drainage bottle 504.

The drainage pump 506 discharges the irrigation fluid inside the joint cavity C1 to the drainage bottle 504 through the flow channel of the drainage tube 505 from the guiding device 4 inserted into the joint cavity C1. Although the first embodiment is explained using the drainage pump 506, it is not limited thereto, and a suction device that is equipped in the facility may be used.

### Configuration of Illumination Device

Next, a configuration of the illumination device 6 will be explained.

The illumination device 6 has two light sources that respectively emit two illumination lights having different wavelengths from each other. The two illumination lights are, for example, a white light that is visible light and an infrared light or special light that is invisible light. From the invisible light illumination, information based on a wavelength different from the visible light can be acquired. For example, in an imaging environment in which turbidity has occurred, an infrared image captured under the infrared light illumination provides an image having a higher contrast than a standard image captured under the white light illumination. Therefore, by extracting an edge component from an image captured under the infrared light illumination, and by superimposing the extracted edge component on the standard image captured under the white light illumination, or a correction image obtained by correcting the standard image, an image in which an edge is further enhanced can be generated. The illumination light from the illumination device 6 is propagated to the endoscope 201 through a light guide, and is irradiated from a distal end of the endoscope 201.

### Functional Configuration of Entire Treatment System

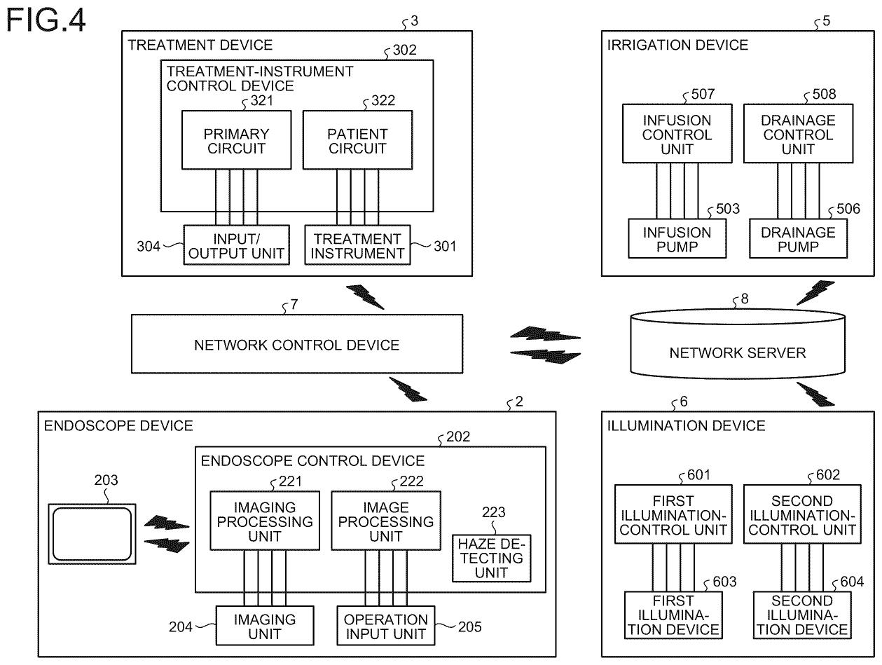

Next, a functional configuration of the entire treatment system will be explained. FIG. 4 is a block diagram illustrating an overview of a functional configuration of the entire treatment system 1.

The treatment system 1 illustrated in FIG. 4 further includes a network control device 7 that controls communications of the entire system, and a network server 8 that stores various kinds of data, in addition to the configuration described above (refer to FIG. 1).

The network control device 7 is connected to the endoscope device 2, the treatment device 3, the irrigation device 5, the illumination device 6, and the network server 8 in a communication-enabled manner. Although an example in which the devices are connected wirelessly is illustrated in FIG. 4, they may be connected wiredly. In the following, a detailed functional configuration of the endoscope device 2, the treatment device 3, the irrigation device 5, and the illumination device 6 will be explained.

The network server 8 is connected to the endoscope device 2, the treatment device 3, the irrigation device 5, the illumination device 6, and the network control device 7 in a communication-enabled manner. The network server 8 stores various kinds of data of the respective devices that constitute the treatment system 1. The network server 8 is constituted of, for example, a processor having hardware, such as a CPU, and a memory, such as a hard disk drive (HDD) and a solid state drive (SSD).

### Functional Configuration of Endoscope Device

Next, a functional configuration of the endoscope device 2 described above will be explained.

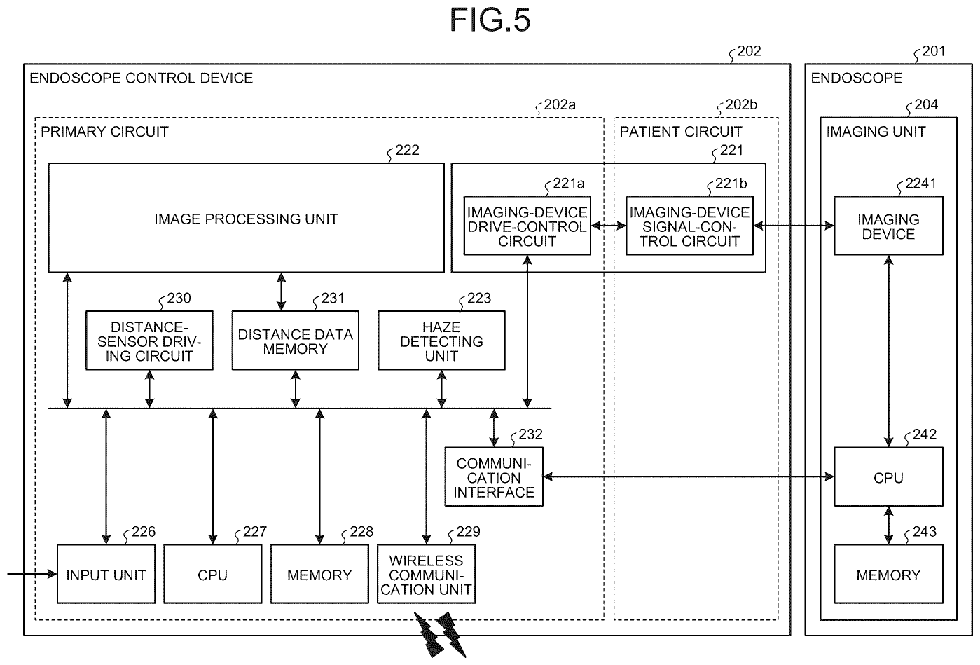

FIG. 5 is a block diagram illustrating a detailed functional configuration of the endoscope device 2.

As illustrated in FIG. 4 and FIG. 5, the endoscope device 2 includes an endoscope control device 202, a display device 203, an imaging unit 204 provided in the endoscope 201, and an operation input unit 205.

The endoscope control device 202 includes an imaging processing unit 221, an image processing unit 222, a haze detecting unit 223, an input unit 226, a CPU 227, a memory 228, a wireless communication unit 229, a distance-sensor driving circuit 230, a distance data memory 231, and a communication interface 232.

The imaging processing unit 221 includes an imaging-device drive-control circuit 221a that controls driving of an imaging device 2241 included in the imaging unit 204 provided in the endoscope 201, and an imaging-device signal-control circuit 221b that performs signal control of the imaging device 2241 provided in a patient circuit 202b that is electrically insulated from a primary circuit 202a. The imaging-device drive-control circuit 221a is arranged in the primary circuit 202a. Moreover, the imaging-device signal-control circuit 221b is arranged in the patient circuit 202b that is electrically insulated from the primary circuit 202a.

The image processing unit 222 performs predetermined image processing with respect to input image data (RAW data), to output to the display device 203 through a bus. The image processing unit 222 is constituted of, for example, a processor having hardware, such as a digital signal processor (DSP) or a field programmable gate array (FPGA). The image processing unit 222 loads a program stored in the memory 228 to a work area of a memory to execute, and implements a functional module that meets a predetermined purpose by causing hardware and software to cooperate by controlling the respective components and the like through execution of the program by the processor. A detailed functional configuration of the image processing unit 222 will be described later.

The haze detecting unit 223 detects a haze in the field of view of the endoscope 201 inside the joint cavity C1 based on information relating to haze in the field of view of the endoscope 201. The information relating to haze includes, for example, a value acquired from image data generated by the endoscope 201, physical properties (turbidity) of the irrigation fluid, an impedance acquired from the treatment device 3, and the like.

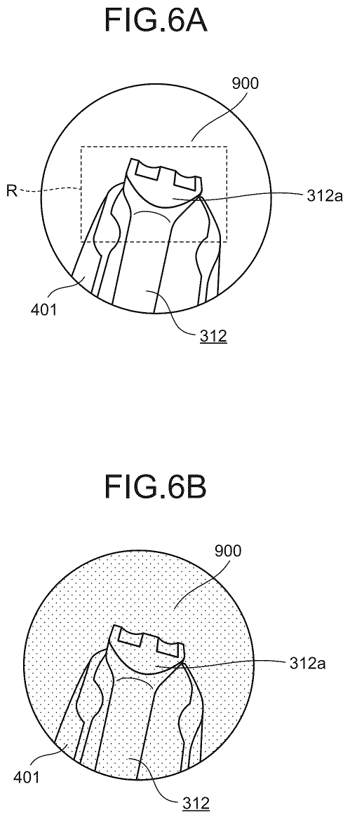

FIG. 6A is a diagram illustrating a state in which the field of view of the endoscope 201 is in a good condition.

FIG. 6B is a diagram illustrating a state in which the field of view of the endoscope 201 is in a poor condition.

Each of FIG. 6A and FIG. 6B schematically illustrates a display image corresponding to image data, which is the field of view of the endoscope 201, when a bone hole is formed in a femoral condyle 900 by an operator. Out of these, FIG. 6B is a schematic illustration of a state in which the field of view of the endoscope 201 is cloudy due to bone crushed into minute particles by driving of the ultrasound probe 312. In FIG. 6B, the minute bone is expressed by a dot.

The input unit 226 accepts input of a signal input by the operation input unit 205 and input of signals from the respective devices constituting the treatment system 1.

The CPU 227 oversees and controls the operation of the endoscope control device 202. The CPU 227 loads a program stored in the memory 228 to a work area of a memory to execute, and controls operation of the respective components of the endoscope control device 202 by causing hardware and software to cooperate by controlling the respective components and the like through execution of the program by the processor.

The memory 228 stores various kinds of information necessary for the operation of the endoscope control device 202, various kinds of programs to be executed by the endoscope control device 202, image data acquired by the imaging unit 204, and the like. The memory 228 is constituted of, for example, a random access memory (RAM), a read only memory (ROM), a frame memory, and the like.

The wireless communication unit 229 is an interface to perform wireless communication with other devices. The wireless communication unit 229 is constituted of, for example, a communication module supporting Wi-Fi (registered trademark), Bluetooth (registered trademark), or the like.

The distance-sensor driving circuit 230 drives a not illustrated distance sensor that measures a distance to a predetermined object in an image captured by the imaging unit 204. In the first embodiment, the distance sensor may be arranged in the imaging device 2241. In this case, the imaging device 2241 may implement phase difference pixels capable of measuring a distance to the predetermined object from the imaging device 2241 in place of effective pixels. A time of flight (ToF) sensor may, of course, be provided near the distal end of the endoscope 201.

The distance data memory 231 stores distance data detected by the distance sensor. The distance data memory 231 is constituted of, for example, a RAM, a ROM, and the like.

The communication interface 232 is an interface to perform communication with the imaging unit 204.

The components described above except the imaging-device signal-control circuit 221b are arranged in the primary circuit 202a, and are connected to one another through a bus wiring.

The imaging unit 204 is arranged in the endoscope 201. The imaging unit 204 includes a imaging device 2241, a CPU 242, and a memory 243.

The imaging device 2241 generates image data by imaging a subject image that is formed by one or more optical systems not illustrated, and outputs the generated image data to the endoscope control device 202 under the control of the CPU 242. The imaging device 2241 is constituted of an imaging sensor, such as a charge coupled device (CCD) or a complementary metal oxide semiconductor (CMOS).

The CPU 242 oversees and controls the operation of the imaging unit 204. The CPU 242 loads a program stored in the memory 243 to a work area of a memory to execute, and controls the operation of the imaging unit 204 by causing hardware and software to cooperate by controlling the respective components and the like through execution of the program by the processor.

The memory 243 stores various kinds of information necessary for the operation of the imaging unit 204, various kinds of programs to be executed by the endoscope 201, image data generated by the imaging unit 204, and the like. The memory 243 is constituted of a RAM, a ROM, a frame memory, and the like.

The operation input unit 205 is composed of an input interface, such as a mouse, a keyboard, a touch panel, and a microphone, and accepts operation input of the endoscope device 2 by an operator.

### Functional Configuration of Treatment Device

Next, a functional configuration of the treatment device 3 will be explained.

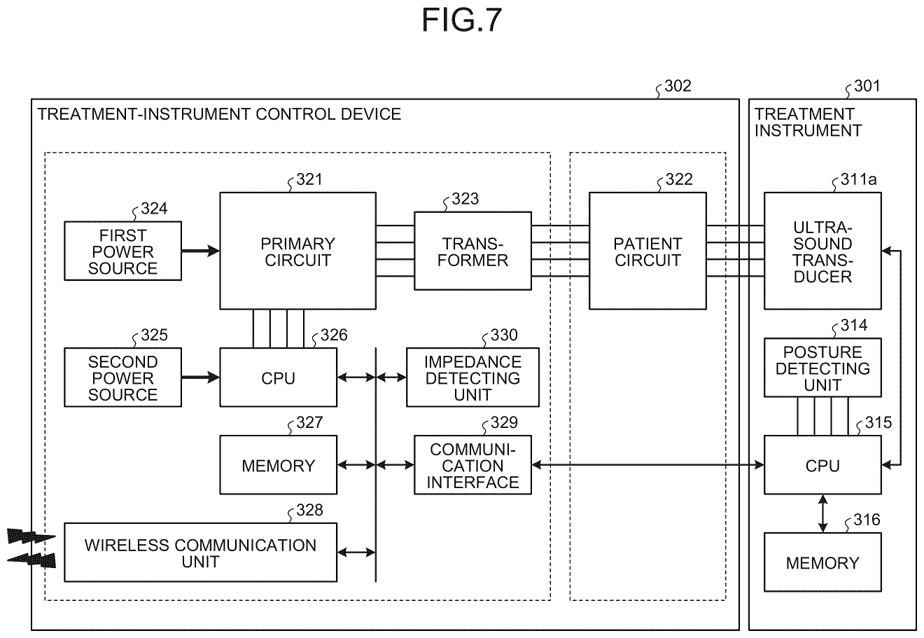

FIG. 7 is a block diagram illustrating a detailed functional configuration of the treatment device 3.

As illustrated in FIG. 4 and FIG. 7, the treatment device 3 includes a treatment instrument 301, a treatment-instrument control device 302, and an input/output unit 304.

The treatment instrument 301 includes an ultrasound transducer 312a, a posture detecting unit 314, a CPU 315, and a memory 316.

The posture detecting unit 314 detects a position of the treatment instrument 301, and outputs this detection result to the CPU 315. The posture detecting unit 314 is constituted of at least one of an acceleration sensor and angular velocity sensor.

The CPU 315 oversees and controls the operation of the treatment instrument 301 including the ultrasound transducer 312a. The CPU 315 loads a program stored in the memory 316 to a work area of a memory to execute, and implements a functional module that meets a predetermined purpose by causing hardware and software to cooperate by controlling the respective components and the like through execution of the program by the processor.

The memory 316 stores various kinds of information necessary for the operation of the treatment instrument 301, various kinds of programs to be executed by the treatment instrument 301, identification information for identifying a type, a manufacturing date, performance, and the like of the treatment instrument 301.

The treatment-instrument control device 302 includes a primary circuit 321, a patient circuit 322, a transformer 323, a first power source 324, a second power source 325, a CPU 326, a memory 327, a wireless communication unit 328, a communication interface 329, and an impedance detecting unit 330.

The primary circuit 321 generates supply power to the treatment instrument 301.

The patient circuit 322 is electrically insulated from the primary circuit 321. The transformer 323 electromagnetically connects the primary circuit 321 and the patient circuit 322.

The first power source 324 is a high voltage power source that supplies driving power to the treatment instrument 301.

The second power source 325 is a low voltage power source that supplies driving power of a control circuit in the treatment-instrument control device 302.

The CPU 326 oversees and controls the operation of the treatment-instrument control device 302. The CPU 326 loads a program stored in the memory 327 to a work area of a memory to execute, and controls operation of the respective components of the treatment-instrument control device 302 by causing hardware and software to cooperate by controlling the respective components and the like through execution of the program by the processor.

The memory 327 stores various kinds of information necessary for the operation of the treatment-instrument control device 302, various kinds of programs to be executed by the treatment-instrument control device 302, and the like. The memory 327 is constituted of a RAM, a ROM, and the like.

The wireless communication unit 328 is an interface to perform wireless communication with other devices. The wireless communication unit 328 is constituted of, for example, a communication module supporting Wi-Fi (registered trademark), Bluetooth (registered trademark), or the like.

The communication interface 329 is an interface to perform communication with the treatment instrument 301.

The impedance detecting unit 330 detects an impedance at the time of driving of the treatment instrument 301, and outputs this detection result to the CPU 326. Specifically, the impedance detecting unit 330 is electrically connected, for example, between the first power source 324 and the primary circuit 321, detects an impedance of the treatment instrument 301 based on a frequency of the first power source 324, and outputs this detection result to the CPU 326.

The input/output unit 304 is constituted of an input interface, such as a mouse, a keyboard, a touch panel, and a microphone, and an output interface, such as a monitor and a speaker, and outputs operation input of the endoscope device 2 by an operator, and various kinds of information to notify to the operator.

### Functional Configuration of Irrigation Device

Next, a functional configuration of the irrigation device 5 will be explained.

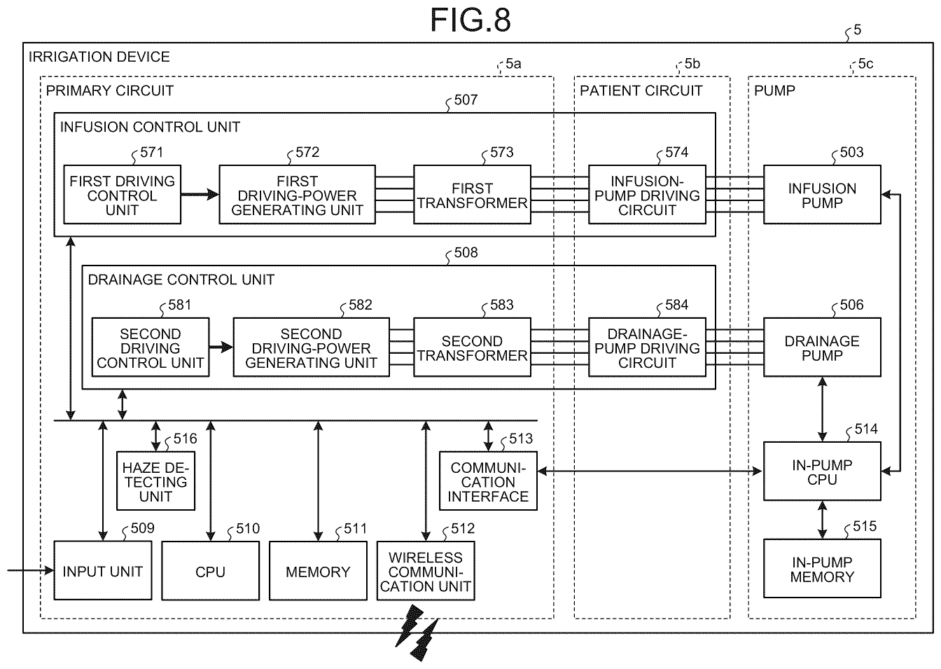

FIG. 8 is a block diagram illustrating a detailed functional configuration of the irrigation device 5.

As illustrated in FIG. 4 and FIG. 8, the irrigation device 5 includes the infusion pump 503, the drainage pump 506, an infusion control unit 507, a drainage control unit 508, an input unit 509, a CPU 510, a memory 511, a wireless communication unit 512, a communication interface 513, an in-pump CPU 514, an in-pump memory 515, and a haze detecting unit 516.

The infusion control unit 507 includes a first driving control unit 571, a first driving-power generating unit 572, a first transformer 573, and an infusion-pump driving circuit 574.

The first driving control unit 571 controls driving of the first driving-power generating unit 572 and the infusion-pump driving circuit 574.

The first driving-power generating unit 572 generates driving power of the infusion pump 503, and supplies this driving power to the first transformer 573.

The first transformer 573 electromagnetically connects the first driving-power generating unit 572 and the infusion-pump driving circuit 574.

In the infusion control unit 507 thus configured, the first driving control unit 571, the first driving-power generating unit 572, and the first transformer 573 are arranged in a primary circuit 5a. Moreover, the infusion-pump driving circuit 574 is arranged in a patient circuit 5b that is electrically insulated from the primary circuit 5a.

The drainage control unit 508 includes a second driving control unit 581, a second driving-power generating unit 582, a second transformer 583, and a drainage-pump driving circuit 584.

The second driving control unit 581 controls driving of the second driving-power generating unit 582 and the drainage-pump driving circuit 584.

The second driving-power generating unit 582 generates driving power of the drainage pump 506, and supplies the generated driving power to the second transformer 583.

The second transformer 583 electromagnetically connects the second driving-power generating unit 582 and the drainage-pump driving circuit 584.

In the drainage control unit 508 thus configured, the second driving control unit 581, the second driving-power generating unit 582, and the second transformer 583 are arranged in the primary circuit 5a. Moreover, the drainage-pump driving circuit 584 is arranged in the patient circuit 5b that is electrically insulated from the primary circuit 5a.

The input unit 509 accepts input of signals from the respective devices constituting the treatment system 1, and outputs the accepted signal to the CPU 510 and the in-pump CPU 514.

The CPU 510 and the in-pump CPU 514 oversees and controls the operation of the irrigation device 5 in collaboration. The CPU 510 loads a program stored in the memory 511 to a work area of a memory to execute, and controls operation of the respective components of the irrigation device 5 by causing hardware and software to cooperate by controlling the respective components and the like through execution of the program by the processor.

The memory 511 stores various kinds of information necessary for the operation of the irrigation device 5 and various kinds of programs executed by the irrigation device 5. The memory 511 is constituted of a RAM, a ROM, and the like.

The wireless communication unit 512 is an interface to perform wireless communication with other devices. The wireless communication unit 512 is constituted of, for example, a communication module supporting Wi-Fi, Bluetooth, or the like.

The communication interface 513 is an interface to perform communication with the infusion pump 503 and the endoscope 201.

The in-pump memory 515 stores various kinds of information necessary for the operation of the infusion pump 503 and the drainage pump 506, and various kinds of programs executed by the infusion pump 503 and the drainage pump 506.

The haze detecting unit 516 detects turbidity of the irrigation fluid based on at least one of physical properties, an absorbance, an impedance, and a resistance value of the irrigation fluid flowing inside the drainage tube 505, and outputs this detection result to the CPU 510.

In the irrigation device 5 thus configured, the input unit 509, the CPU 510, the memory 511, the wireless communication unit 512, the communication interface 513, and the haze detecting unit 516 are arranged in the primary circuit 5a. Furthermore, the in-pump CPU 514 and the in-pump memory 515 are arranged in a pump 5c. The in-pump CPU 514 and the in-pump memory 515 may be arranged near the infusion pump 503, or may be arranged near the drainage pump 506.

### Functional Configuration of Illumination Device

Next, a functional configuration of the illumination device 6 will be explained.

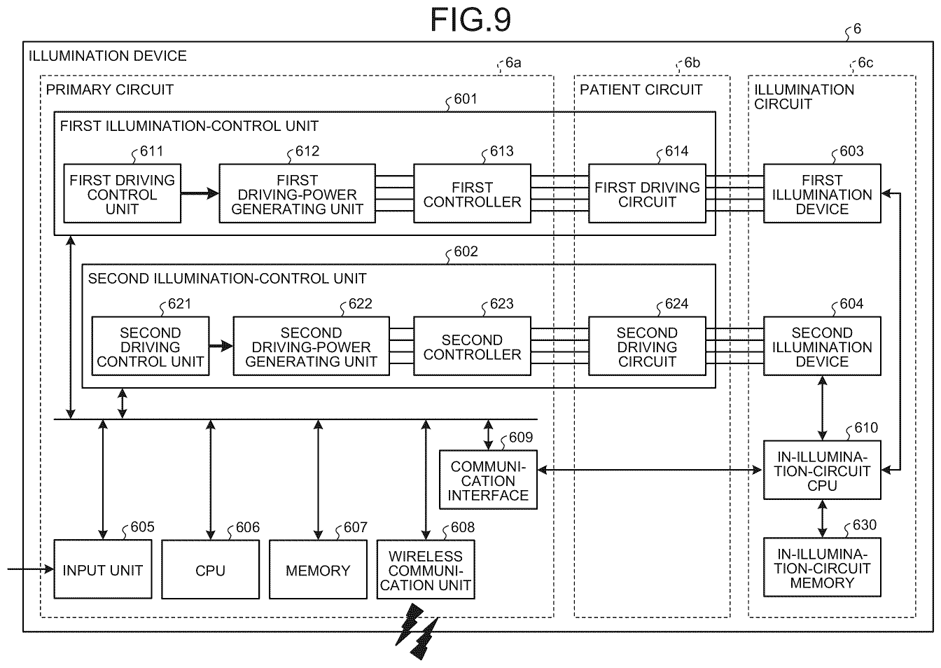

FIG. 9 is a block diagram illustrating a detailed functional configuration of the illumination device 6.

As illustrated in FIG. 4 and FIG. 9, the illumination device 6 includes a first illumination-control unit 601, a second illumination-control unit 602, a first illumination device 603, a second illumination device 604, an input unit 605, a CPU 606, a memory 607, a wireless communication unit 608, a communication interface 609, an in-illumination-circuit CPU 610, and an in-illumination-circuit memory 630.

The first illumination-control unit 601 includes a first driving control unit 611, a first driving-power generating unit 612, a first controller 613, and a first driving circuit 614.

The first driving control unit 611 controls driving of the first driving-power generating unit 612, the first controller 613, and the first driving circuit 614.

The first driving-power generating unit 612 generates driving power of the first illumination device 603, and outputs this driving power to the first controller 613 under control of the first driving control unit 611.

The first controller 613 controls light output of the first illumination device 603 by controlling the first driving circuit 614 according to the driving power input from the first driving-power generating unit 612.

The first driving circuit 614 drives the first illumination device 603 under the control of the first controller 613 to output the illumination light.

In the first illumination-control unit 601 thus configured, the first driving control unit 611, the first driving-power generating unit 612, and the first controller 613 are arranged in a primary circuit 6a. Moreover, the first driving circuit 614 is arranged in a patient circuit 6b that is electrically insulated from the primary circuit 6a.

The second illumination-control unit 602 includes a second driving control unit 621, a second driving-power generating unit 622, a second controller 623, and a second driving circuit 624.

The second driving control unit 621 controls the second driving-power generating unit 622, the second controller 623, and the second driving circuit 624.

The second driving-power generating unit 622 generates driving power of the second illumination device 604, and outputs this driving power to the second controller 623 under the control of the second driving control unit 621.

The second controller 623 controls light output of the second illumination device 604 by controlling the second driving circuit 624 according to the driving power input from the second driving-power generating unit 622.

The second driving circuit 624 drives the second illumination device 604, and outputs illumination light under the control of the second controller 623.

In the second illumination-control unit 602 thus configured, the second driving control unit 621, the second driving-power generating unit 622, and the second controller 623 are arranged in the primary circuit 6a. Moreover, the second driving circuit 624 is arranged in the patient circuit 6b that is electrically insulated from the primary circuit 6a.

The first illumination device 603 irradiates light in a wavelength range of visible light (hereinafter, simply “visible light”) to the subject as the first illumination light to illuminate the subject through the endoscope 201. The visible light is white light (wavelength band λ=380 nm to 780 nm). The first illumination device 603 is constituted of, for example, a white light emitting diode (LED) lamp, a halogen lamp, or the like.

The second illumination device 604 irradiates light in a wavelength range outside visible light (hereinafter, simply “invisible light”) to the subject as the second illumination light to illuminate the subject through the endoscope 201. The invisible light is infrared light (wavelength band λ=780 nm to 2500 nm). The second illumination device 604 is constituted of, for example, an infrared LED lamp, or the like.

The input unit 605 accepts input of a signal from the respective devices constituting the treatment system 1, and outputs the accepted signal to the CPU 606 and the in-illumination-circuit CPU 610.

The CPU 606 and the in-illumination-circuit CPU 610 oversee and control the operation of the illumination device 6 in corporation. The CPU 606 loads a program stored in the memory 607 to a work area of a memory to execute, and controls operation of the respective components of the illumination device 6 by causing hardware and software to cooperate by controlling the respective components and the like through execution of the program by the processor.

The memory 607 stores various kinds of information necessary for the operation of the illumination device 6, and various kinds of programs to be executed by the illumination device 6. The memory 607 is constituted of, for example, a RAM, a ROM, and the like.

The wireless communication unit 608 is an interface to perform wireless communication with other devices. The wireless communication unit 608 is constituted of, for example, a communication module supporting Wi-Fi, Bluetooth, or the like.

The communication interface 609 is an interface to perform communication with an illumination circuit 6c.

The in-illumination circuit memory 630 stores various kinds of information and a program necessary for the operation of the first illumination device 603 and the second illumination device 604. The in-illumination circuit memory 630 is constituted of a RAM, a ROM, and the like.

In the illumination device 6 thus configured, the input unit 605, the CPU 606, the memory 607, the wireless communication unit 608, and the communication interface 609 are arranged in the primary circuit 6a. Furthermore, the first illumination device 603, the second illumination device 604, the in-illumination-circuit CPU 610, and an in-illumination-circuit memory 61A are arranged in the illumination circuit 6c.

### Detailed Configuration of Image Processing Unit

Next, a detailed configuration of the image processing unit 222 described above will be explained.

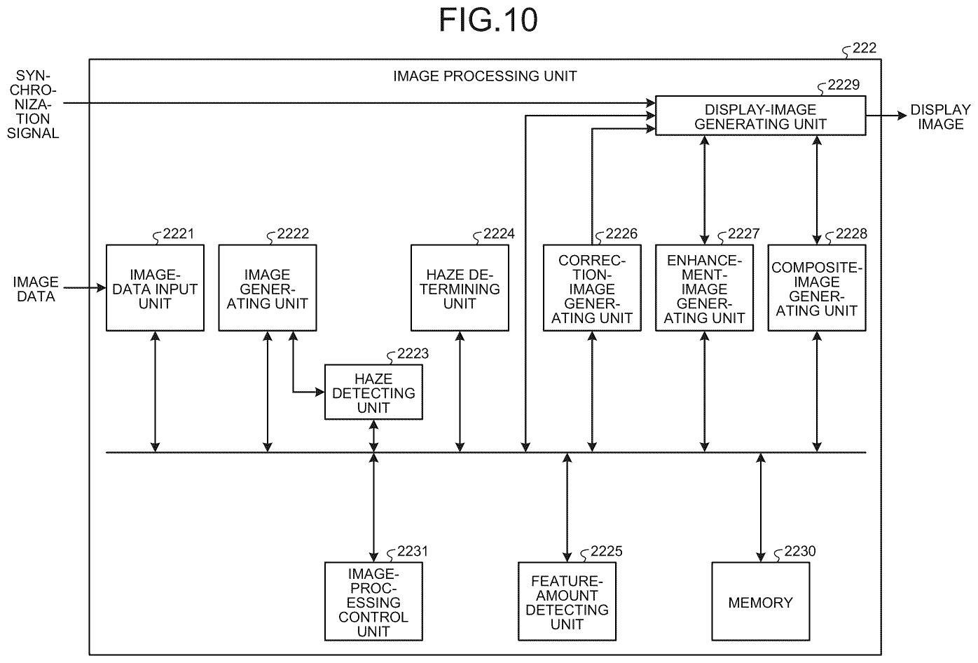

FIG. 10 is a block diagram illustrating a detailed functional configuration of the image processing unit 222.

The image processing unit 222 illustrated in FIG. 10 includes an image-data input unit 2221, an image generating unit 2222, a haze detecting unit 2223, a haze determining unit 2224, a feature-amount detecting unit 2225, a correction-image generating unit 2226, an enhancement-image generating unit 2227, a composite-image generating unit 2228, a display-image generating unit 2229, a memory 2230, and an image-processing control unit 2231.

The image-data input unit 2221 accepts input of image data generated by the endoscope 201 and input of a signal from the respective devices constituting the treatment system 1, and outputs the accepted data and the signal to the bus.

The image generating unit 2222 performs predetermined image processing with respect to the image data (RAW data) input through the image-data input unit 2221 to generate color image data (hereinafter, simply “standard image data”) in accordance with a synchronization signal synchronized with imaging drive of the imaging unit 204, and outputs this standard image data to the haze detecting unit 2223, the feature-amount detecting unit 2225, the correction-image generating unit 2226, and the display-image generating unit 2229. Specifically, the image generating unit 2222 generates the standard image data based on pixel values of the R pixel, the G pixel, and the B pixel included in the image data. The predetermined image processing includes, for example, demosaicing processing, color correction processing, black-level correction processing, noise reduction processing, and y correction processing, and the like. In the first embodiment, the image generating unit 2222 functions as an image acquiring unit that acquires image data including a region in which a living body is treated with the energy treatment instrument, such as the ultrasound probe 312. The image generating unit 2222 may generate the standard image data based on a driving signal of the treatment instrument 301.

The haze detecting unit 2223 detects changes in tone from at least a part of the standard image corresponding to the standard image data (hereinafter, simply “standard image”) based on the standard image data generated by the image generating unit 2222, and outputs this detection result to the haze determining unit 2224. Specifically, the haze detecting unit 2223 detects haze in the field of view of the endoscope 201 as at least a part of the region in the standard image based on the standard image generated by the image generating unit 2222, and outputs this detection result to the haze determining unit 2224. As for the detecting method of haze by the haze detecting unit 2223, detailed explanation of the detecting method is omitted because it is detected by a similar method to the haze component detection of a haze estimating unit 2226a of the correction-image generating unit 2226 described later.

The haze of the field of view of the endoscope 201 is a degree of turbidity caused by bone powder or debris dissolved in the irrigation fluid that is a cause of deterioration in tones. As the cause of deterioration of the image quality, in addition to a phenomenon caused by dissolution of living tissues, such as bone powder, debris, blood, and bone marrow, into the irrigation fluid, a phenomenon of smoke and sparks during a procedure by the treatment instrument 301 is also considered. In the following, a state in which the irrigation fluid has become turbid because of dissolution of bone powder into the irrigation fluid will be explained. Because the irrigation fluid in which a living tissue has dissolved becomes turbid and opaque in white overall, it has characteristics of having high brightness, low saturation (poor color reproduction), and low contrast. Therefore, the haze detecting unit 2223 detects the haze (haze components) of the field of view of the endoscope 201 by calculating contrast, brightness, and saturation as the haze of the field of view of the endoscope 201 for each pixel constituting the first image.

The haze determining unit 2224 determines whether the haze detected by the haze detecting unit 2223 is equal to or larger than a predetermined value, and outputs this determination result to the image-processing control unit 2231. The predetermined value is, for example, a value corresponding to a level at which a treatment site in the field of view of the endoscope 201 becomes obscured by the haze. For example, as a value of the level at which a treatment site becomes obscured is a value of high brightness and low saturation (high-brightness white.

The feature-amount detecting unit 2225 detects a region including at least a part of the treatment instrument 301, which is the energy treatment instrument, from the standard image generated by the image generating unit 2222, and outputs this detection result to the enhancement-image generating unit 2227. The feature-amount detecting unit 2225 detects a feature amount of each pixel from the standard image generated by the image generating unit 2222, and detects a region including at least a part of the treatment instrument, which is the energy treatment instrument based on this feature amount. The feature amount includes, for example, an edge component, brightness component, and the like. The feature-amount detecting unit 2225 may detect the treatment instrument 301 captured in the standard image by using a technique of a publicly-known pattern matching processing.

The correction-image generating unit 2226 generates correction image data by performing tone correction with respect to the standard image input from the image generating unit 2222 based on the detection result input from the haze detecting unit 2223 in accordance with the synchronization signal synchronized with imaging drive of the imaging unit 204, and outputs a correction image corresponding to this correction image data (hereinafter, simply, “correction image”) to the enhancement-image generating unit 2227, the composite-image generating unit 2228, and the display-image generating unit 2229. Specifically, the correction-image generating unit 2226 generates a correction image obtained by removing a haze (haze component) included in the standard image, and outputs this correction image to the enhancement-image generating unit 2227, the composite-image generating unit 2228, or the display-image generating unit 2229. Details of the correction-image generating unit 2226 will be described later.

The enhancement-image generating unit 2227 generates enhancement image data by performing the edge enhancement processing on the standard image input from the image generating unit 2222 or the correction image input from the correction-image generating unit 2226, based on the synchronization signal synchronized with the imaging drive of the imaging unit 204 and the detection result of the feature-amount detecting unit 2225, and outputs this enhancement image data (hereinafter, simply “enhancement image”) to the composite-image generating unit 2228 or the display-image generating unit 2229. Specifically, the enhancement-image generating unit 2227 performs edge extraction to extract an edge component with respect to a region of the standard image or the correction image that includes at least a part of the treatment instrument 301, which is the energy treatment instrument, detected by the feature-amount detecting unit 2225, and generates an enhancement image in which edge enhancement processing to enhance an edge compared to other regions is performed on this extracted edge component.

The composite-image generating unit 2228 generates composite image data by combining the standard image input from the image generating unit 2222 or the correction image input from the correction-image generating unit 2226, and the enhancement image input from the enhancement-image generating unit 2227 at a predetermined ratio according to the synchronization signal synchronized with the imaging drive of the imaging unit 204 under control of the image-processing control unit 2231, and outputs a composited image corresponding to this composite image data (hereinafter, simply “composite image”) to the display-image generating unit 2229. The predetermine ratio is, for example, 5:5. The composite-image generating unit 2228 may change the ratio of combining the standard image or the correction image and the enhancement image based on the detection result of the haze detecting unit 2223 appropriately, and may change the combining ratio according to a component and a kind of the haze. The composite image may be generated by adding or superimposing an edge component extracted from the enhancement image to or on the standard image or the correction image.

The display-image generating unit 2229 generates a display image corresponding to display data to be displayed on the display device 203 based on at least one of the standard image input from the image generating unit 2222, the correction image input from the correction-image generating unit 2226, the enhancement image input from the enhancement-image generating unit 2227, and the composite image input from the composite-image generating unit 2228 according to the synchronization signal that is synchronized with imaging drive of the imaging unit under control of the image-processing control unit 2231, and outputs it to the display device 203. Specifically, the display-image generating unit 2229 converts the format of an input image into a predetermined format, for example, converting from the RGB format to the YCbCr format, to output to the display device 203. The display-image generating unit 2229 may generate a display image based on a driving signal of the treatment instrument 301.

The memory 2230 stores various kinds of information necessary for the operation of the image processing unit 222, various kinds of programs executed by the image processing unit 222, various kinds of image data, and the like. The memory 2230 is constituted of a RAM, a ROM, a frame memory, and the like.

The image-processing control unit 2231 controls the respective components constituting the image processing unit 222. The image-processing control unit 2231 loads a program stored in the memory 2230 to a work area of a memory to execute, and controls operation of the respective components of the image processing unit 222 by causing hardware and software to cooperate by controlling the respective components and the like through execution of the program by the processor.

### Detailed Functional Configuration of Correction-Image Generating Unit

Next, a detailed functional configuration of the correction-image generating unit 2226 will be explained.

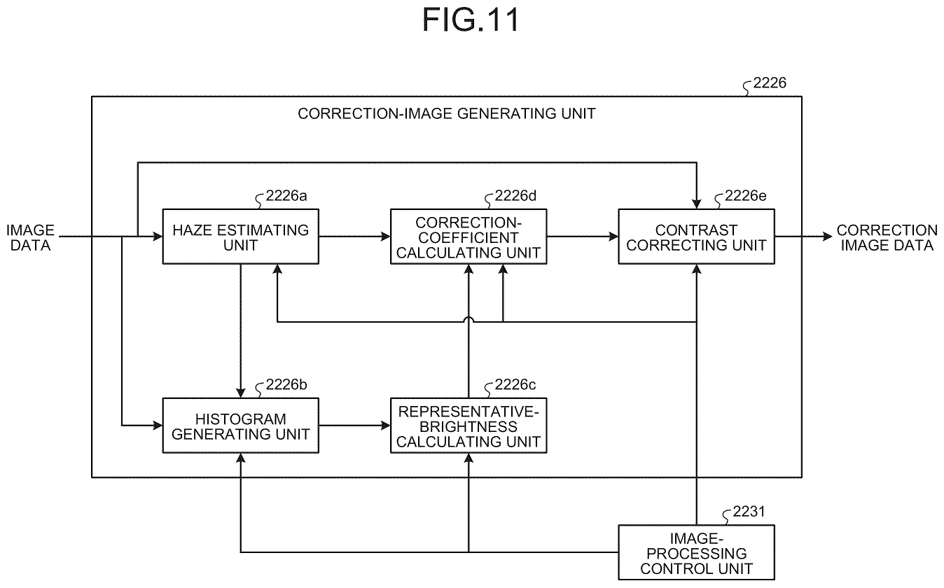

FIG. 11 is a block diagram illustrating a detailed functional configuration of the correction-image generating unit 2226.

The correction-image generating unit 2226 illustrated in FIG. 11 includes the haze estimating unit 2226a, a histogram generating unit 2226b, a representative-brightness calculating unit 2226c, a correction-coefficient calculating unit 2226d, and a contrast correcting unit 2226e.

The haze estimating unit 2226a estimates a haze component of each pixel in the standard image. The haze component of each pixel is a degree of turbidity caused by bone powder or debris dissolved in the irrigation fluid that is a cause of deterioration of the image quality, such as contrast and saturation in the standard image. As the cause of deterioration of the image quality, in addition to a phenomenon caused by dissolution of living tissues, such as bone powder, debris, blood, and bone marrow, into the irrigation fluid, a phenomenon of smoke and sparks during a procedure by the treatment instrument 301 is also considered. In the following, a turbidity in a state in which the irrigation fluid has become turbid because of dissolution of bone powder into the irrigation fluid will be explained. Because the irrigation fluid in which a living tissue has dissolved has characteristics of having high brightness, low saturation (poor color reproduction), and low contrast.

Therefore, the haze estimating unit 2226a estimates a haze component in the field of view of the endoscope 201 by calculating the contrast, or the brightness and saturation of the first image. Specifically, the haze estimating unit 2226a estimates a haze component H(x, y) based on an R value, a G value, and a B value of a pixel at coordinates (x, y) in the first image.

When the R value, the G value, and the B value at the coordinates (x, y) are Ir, Ig, and Ib, respectively, the haze component H(x, y) of the pixel at the coordinates (x, y) is estimated by following Equation (1).

H(x,y)=min(Ir,Ig,Ib) (1)

The haze estimating unit 2226a performs calculation of Equation (1) described above for each pixel of the first image. The haze estimating unit 2226a sets a scan region F (small region) of a predetermined size with respect to the first image. The size of this scan region F is, for example, pixels of a predetermined size, m×n (m, n are positive integers). In the following explanation, the pixel at the center of the scan area F will be referred to as the reference pixel. Furthermore, in the following explanation, the respective pixels around the reference pixel in the scan region F will be referred to as neighboring pixels. Furthermore, in the following explanation, the scan region F will be explained as being formed with a size of, for example, 5×5 pixels. Of course, the scan region F is also applicable to a single pixel.

The haze estimating unit 2226a calculates (Ir, Ig, Ib) of each pixel in the scan region F while shifting the position of the scan region F with respect to the first image, and estimates the minimum values out of those as the haze component H(x, y) of the reference pixel. In the first image, the pixel values in the high-brightness and low-saturation region have the R value, the G value, and the B value that are equal and large and, therefore, values of min (Ir, Ig, Ib) become large. That is, the high-brightness and low-saturation region have large values for the haze component H(x, y).

On the other hand, the pixel values in a low-brightness or high-saturation region have at least one of the R value, the G value, and the B value that is low and, therefore, values of min (Ir, Ig, Ib) become small. That is, the low-brightness or high saturation region have small values for the haze component H(x, y).

As described, the haze component H(x, y) has a larger value as the concentration of bone powder dissolved in the irrigation fluid becomes higher (the whiteness of the bone powder becomes more significant) and a smaller value as the concentration of bone powder becomes lower. In other words, the haze component H(x, y) increases as the color (whiteness) of the irrigation fluid becomes more intense due to the bone powder dissolved in the irrigation fluid, and decreases as the color of the irrigation fluid becomes less intense.

The haze estimating unit 2226a estimates the haze component H(x, y) by using Equation (1) described above, but it is not limited thereto, and any indicator that indicates high brightness and low saturation can be used as the haze component. The haze estimating unit 2226a may use at least one of a local contrast value, an edge strength, a color density, and a subject distance to estimate the haze component. Moreover, the haze detecting unit 2223 described above detects a haze (haze component) by a method similar to that of the haze estimating unit 2226a.

The histogram generating unit 2226b determines a distribution of a histogram in a local region including the reference pixel of the first image and the neighboring pixels of this reference pixel based on the haze component H(x, y) input from the haze estimating unit 2226a. This degree of change in the haze component H(x, y) becomes an indicator for determining a region to which each pixel belongs in the local region. Specifically, this degree of change in the haze component H(x, y) is determined based on a difference in the haze component H(x, y) between the reference pixel and the neighboring pixels within the local region.

That is, the histogram generating unit 2226b generates a brightness histogram for the local region including the neighboring pixels for each reference pixel based on the first image input from the image generating unit 2222 and the haze component H(x, y) input from the haze estimating unit 2226a. Typical generation of a histogram is performed by regarding pixel values in a target local region as brightness values, and by counting the frequency of each pixel value by one.

On the other hand, the histogram generating unit 2226b according to the first embodiment assigns weight to the count values for the pixel values of the neighboring pixels according to the haze components H(x, y) of the reference pixel and the neighboring pixels within the local region. The count value for the pixel values of the neighboring pixels take a value, for example, within a range of 0.0 to 1.0. Moreover, the count value is set to have a smaller value as the difference in the haze component H(x, y) between the reference pixel and the neighboring pixels increases, and is set to have a larger value as the difference in the haze component H(x, y) between the reference pixel and the neighboring pixels decreases. Furthermore, the local region is formed with, for example, a size of 7×7 pixels.

In typical histogram generation, if the histogram is created using only brightness, the brightness of a neighboring pixel having a significant difference from the brightness of the target pixel is also counted in the same way. The local histogram is preferable to be generated according to an image region to which the target pixel belongs.

On the other hand, in generation of the brightness histogram in the first embodiment, the count value for the pixel value of the respective pixels in the local region in the first image data is set according to the difference in the haze component H(x, y) between the reference pixel and the respective neighboring pixels within the local region in the first image data of the haze component H(x, y). Specifically, the count value is calculated, for example, by using a Gaussian function (for example, U.S. Pat. No. 6,720,012 or U.S. Pat. No. 6,559,229, note that the blurriness component is replaced with the haze component) such that the value becomes smaller as the difference in the haze component H(x, y) between the reference pixel and the neighboring pixel increases, and becomes larger as the difference in the haze component H(x, y) between the reference pixel and the neighboring pixel decreases.

The method of calculating the count value by the histogram generating unit 2226b is not limited to the one using a Gaussian function as long as it is possible to set the count value to become smaller as the difference between the reference pixel and the neighboring pixel increases. For example, the histogram generating unit 2226b may calculate the count value using a lookup table or a line approximation table instead of a Gaussian function.

Moreover, the histogram generating unit 2226b may be configured to compare the difference in the value between the reference pixel and the neighboring pixel with a threshold, and to decrease the count value of the neighboring pixel (for example, to 0.0) when it is equal to or larger than the threshold.

Furthermore, the histogram generating unit 2226b is not necessarily required to use the frequency of pixel value as the count value. For example, the histogram generating unit 2226b may use each of the R value, the G value, and the B value as the count value. Moreover, the histogram generating unit 2226b may use the G value for the count value as the brightness value.

The representative-brightness calculating unit 2226c calculates representative brightness based on statistical information of the brightness histogram that is input from the histogram generating unit 2226b. The representative brightness includes the brightness of a low brightness region, the brightness of a high brightness region, and the brightness of an intermediate brightness region within an effective brightness range of the brightness histogram. The brightness of the low brightness region is the minimum brightness in the effective brightness range. The brightness of the high brightness region is the maximum brightness in the effective brightness range. The brightness of the intermediate brightness region is centroid brightness. The minimum brightness is the brightness at which the cumulative frequency is at 5% of the maximum value in a cumulative histogram generated from the brightness histogram. The maximum brightness is the brightness at which the cumulative frequency is at 95% of the maximum value in the cumulative histogram generated from the brightness histogram. The centroid brightness is the brightness at which the cumulative frequency is at 50% of the maximum value in the cumulative histogram generated from the brightness histogram.

Furthermore, the percentages of the cumulative frequency corresponding to the minimum brightness, the maximum brightness, and the centroid brightness, namely 5%, 50%, and 95%, respectively, can be changed appropriately. Moreover, although the brightness of the intermediate brightness region is defined as the centroid brightness in the cumulative histogram, it is not limited thereto, and the centroid brightness is not necessarily required to be calculated from the cumulative frequency. For example, the brightness corresponding to the maximum frequency in the brightness histogram can also be applied as the brightness in the intermediate brightness region.

The correction-coefficient calculating unit 2226d calculates a correction coefficient to correct the contrast in the local region based on the haze component H(x, y) input from the haze estimating unit 2226a and the statistical information input from the representative-brightness calculating unit 2226c. Specifically, when the contrast correction is performed by histogram stretching, the correction-coefficient calculating unit 2226d calculates a coefficient for the histogram stretching by using the centroid brightness and the maximum brightness out of the statistical information.

The histogram stretching is processing to enhance a contrast by expanding an effective brightness range of a histogram (for example, refer to U.S. Pat. No. 6,720,012 or U.S. Pat. No. 6,559,229). The correction-coefficient calculating unit 2226d uses histogram stretching as a method to achieve the contrast correction, but it is not limited thereto. As the method to achieve the contrast correction, for example, histogram equalization may be used.

For example, the correction-coefficient calculating unit 2226d may use a method using a cumulative histogram or a line approximation table, as the method of achieving the histogram equalization. This cumulative histogram accumulates the frequency values of the brightness histogram sequentially.

The contrast correcting unit 2226e performs, with respect to the first image input from the image generating unit 2222, contrast correction of the reference pixel in the first image data based on the haze component H(x, y) input from the haze estimating unit 2226a and the correction coefficient input from the correction-coefficient calculating unit 2226d (for example, refer to U.S. Pat. No. 6,720,012 or U.S. Pat. No. 6,559,229).

The correction-image generating unit 2226 thus configured estimates the haze component H(x, y) based on the first image, calculates the brightness histogram and the representative brightness using this estimation result, calculates the correction coefficient to correct the contrast in the local region, and performs the contrast correction based on the haze component H(x, y) and the correction coefficient. The correction-image generating unit 2226 can thereby generate the first correction image obtained by removing the haze from the first image.

### Overview of Treatment

Next, an overview of a treatment performed by an operator by using the treatment system 1 will be explained.

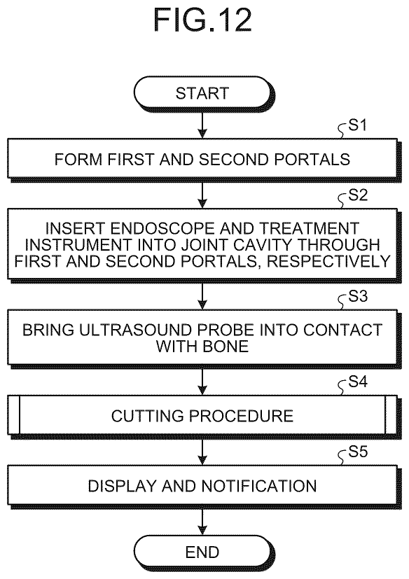

FIG. 12 is a flowchart explaining the overview of the treatment performed by an operator using the treatment system 1. The operator that performs the treatment may be a single surgeon or may be two or more operators including a surgeon and an assistant.

As illustrated in FIG. 12, the operator forms the first portal P1 and the second portal P2 that respectively connect the inside of the joint cavity C1 of the knee joint J1 and the outside of the skin (step S1).

Subsequently, the operator inserts the endoscope 201 into the joint cavity C1 through the first portal P1, inserts the guiding device 4 into the joint cavity C1 through the second portal P2, and inserts the treatment instrument 301 into the joint cavity C1, guided by the guiding device 4 (step S2). Although a case in which the endoscope 201 and the treatment instrument 301 are inserted into the joint cavity C1 through the first portal P1 and the second portal P2 after forming the two portals has been explained herein, the second portal P2 may be formed to insert the guiding device 4 and the treatment instrument 301 into the joint cavity C1 after the first portal P1 is formed and the endoscope 201 is inserted into the joint cavity C1.

Thereafter, the operator brings the ultrasound probe 312 into contact with a bone to be treated while visually confirming the endoscopic image within the joint cavity C1 displayed on the display device 203 (step S3).

Subsequently, the operator performs a cutting procedure using the treatment instrument 301 while viewing the endoscopic image displayed on the display device 203 (step S4). Details of the processing of the treatment system 1 in the cutting procedure will be described later.

Thereafter, the display device 203 performs display and notification processing for displaying the inside of the joint cavity C1 and information relating to a state after the cutting procedure (step S5). The power of the endoscope control device 202 is turned off, for example, after the display and notification processing is performed. The operator ends the treatment using the treatment system 1.

### Details of Cutting Procedure

Next, details of the cutting procedure at step S4 in FIG. 12 described above will be explained.

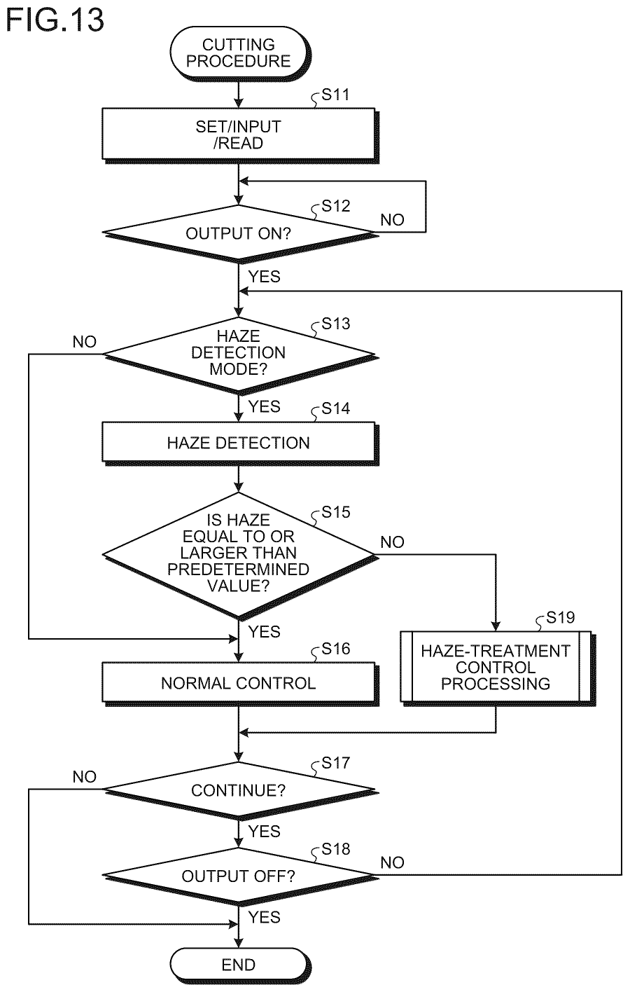

FIG. 13 explains an overview of the processing performed in the cutting procedure by the endoscope control device 202.

In the following, each processing is explained to be performed under control of CPUs of the respective control devices, but the processing may be performed collectively by either one of the control devices, such as the network control device 7.

As illustrated in FIG. 13, the CPU 227 performs communication with the respective devices, and performs setting of control parameters for each of the treatment device 3 and the irrigation device 5, and input of the control parameters for each of the treatment device 3 and the irrigation device 5 (step S11).

Subsequently, the CPU 227 determines whether the respective devices constituting the treatment system 1 have become an output ON state (step S12). When it is determined that the devices of the respective components constituting the treatment system 1 have become the output ON state by the CPU 227 (step S12: YES), the endoscope control device 202 shifts to step S13 described later. On the other hand, when it is determined that the devices of the respective components constituting the treatment system 1 have not become the output ON state by the CPU 227 (step S12: NO), the CPU 227 continues this determination until the devices of the respective components constituting the treatment system 1 become the output ON state.

At step S13, the CPU 227 determines whether the observation mode of the endoscope control device 202 in the treatment system 1 is set to the haze detection mode. When it is determined that the observation mode of the endoscope control device 202 in the treatment system 1 is set to the haze detection mode by the CPU 227 (step S13: YES), the endoscope control device 202 shifts to step S14 described later. On the other hand, when it is determined that the observation mode of the endoscope control device 202 in the treatment system 1 is not set to the haze detection mode by the CPU 227 (step S13: NO), the endoscope control device 202 shifts to step S16 described later.

At step S14, the haze detecting unit 223 detects haze in the field of view of the endoscope 201 based on either one of the standard image generated by the endoscope 201, the detection result of the impedance detecting unit 330 of the treatment-instrument control device 302, and the detection result of the haze detecting unit 516 of the irrigation device 5. Specifically, the haze detecting unit 223 detects the haze in the field of view of the endoscope 201 by using either one of the brightness and the contrast of the standard image when the standard image generated by the endoscope is used. Moreover, the haze detecting unit 223 detects the haze in the field of view of the endoscope 201 based on a change rate of impedance when the impedance detected by the impedance detecting unit 330 of the treatment-instrument control device 302 is used. Furthermore, the haze detecting unit 223 detects the haze in the field of view of the endoscope 201 based on turbidity of the irrigation fluid detected by the haze detecting unit 516 of the irrigation device 5 when the detection result of the haze detecting unit 516 of the irrigation device 5 is used.

Subsequently, the CPU 227 determines whether the haze in the field of view of the endoscope 201 is equal to or larger than a predetermined value based on the detection result detected by the haze detecting unit 223 (step S15).

Specifically, the CPU 227 determines whether an average value of a sum of luminance of respective pixels in the standard image detected by the haze detecting unit 223 is equal to or larger than the predetermined value when the haze detecting unit 223 uses the standard image. The predetermined value as the luminance is a high brightness value close to white. In this case, the CPU 227 determines that there is a haze in the field of view of the endoscope 201 when the average value of the sum of the luminance of the respective pixels in the standard image detected by the haze detecting unit 223 is equal to or larger than the predetermined value. On the other hand, the CPU 227 determines that there is no haze in the field of view of the endoscope 201 when the average value of the luminance of the respective pixels in the standard image detected by the haze detecting unit 223 is equal to or larger than the predetermined value.

Moreover, the CPU 227 determines whether the impedance is equal to or larger than a predetermined value when the haze detecting unit 223 uses the impedance detected by the impedance detecting unit 330. The CPU 227 determines that there is a haze in the field of view of the endoscope 201, when the haze detecting unit 223 determines that the impedance detected by the impedance detecting unit 330 is equal to or larger than the predetermined value. On the other hand, when the impedance detected by the impedance detecting unit 330 is not equal to or larger than the predetermined value, the haze detecting unit 223 determines that there is no haze in the field of view of the endoscope 201.

Furthermore, the CPU 227 determines whether the turbidity of the irrigation fluid is equal to or larger than a predetermined value when the haze detecting unit 223 uses the turbidity of the irrigation fluid detected by the haze detecting unit 516 of the irrigation device 5. When the turbidity of the irrigation fluid detected by the haze detecting unit 223 is equal to or larger than a predetermined value, the CPU 227 determines that there is a haze in the field of view of the endoscope 201. On the other hand, when the turbidity of the irrigation fluid detected by the haze detecting unit 223 is not equal to or larger than the predetermined value, it is determined that there is no haze in the field of view of the endoscope 201.

At step S15, when it is determined that there is a haze in the field of view of the endoscope 201 by the CPU 227 (at step S15: YES), the endoscope control device 202 shifts to step S19 described later. On the other hand, when it is determined that there is no haze in the field of view of the endoscope 201 by the CPU 227 (step S15: NO), the endoscope control device 202 shifts to step S16 described later.

At step S16, the CPU 227 performs normal control with respect to the endoscope control device 202. Specifically, the CPU 227 outputs the standard image (color image) generated by the image processing unit 222 to the display device 203 to display. Thus, the operator can perform treatment by using the treatment instrument 301 while viewing the standard image displayed on the display device 203.

Subsequently, the CPU 227 determines whether the treatment for the subject is being continued by the operator (step S17). Specifically, the CPU 227 determines whether power is being supplied to the treatment instrument 301 by the treatment-instrument control device 302, and determines that the operator is continuing the treatment to the subject when the power is being supplied to the treatment instrument 301 by the treatment-instrument control device 302, and determines that the operator is not continuing the treatment to the subject when it is determined that the power is not being supplied to the treatment instrument 301 by the treatment-instrument control device 302. When the CPU determines that the treatment to the subject is being continued by the operator (step S17: YES), the endoscope control device 202 shifts to step S18 described later. On the other hand, when the CPU 227 determines that the treatment to the subject is not being continued by the operator (step S17: NO), the endoscope control device 202 ends this processing.

At step S18, the CPU 227 determines whether the devices of the respective components constituting the treatment system 1 has become an output OFF state. When it is determined that the devices of the respective components constituting the treatment system 1 has become the output OFF state by the CPU 227 (step S18: YES), the endoscope control device 202 ends this processing. On the other hand, when it is determined that the devices of the respective components constituting the treatment system 1 has not become the output OFF state by the CPU 227 (step S18: NO), the endoscope control device 202 returns to step S13 described above.

At step S19, the endoscope control device 202 performs haze-treatment control processing with respect to the haze in the field of view of the endoscope 201. Details of the haze-treatment control processing will be described later. After step S19, the endoscope control device 202 shifts to step S17.

### Details of Haze-Treatment Control Processing

Next, details of the haze-treatment control processing explained at step S19 in FIG. 13 will be explained.

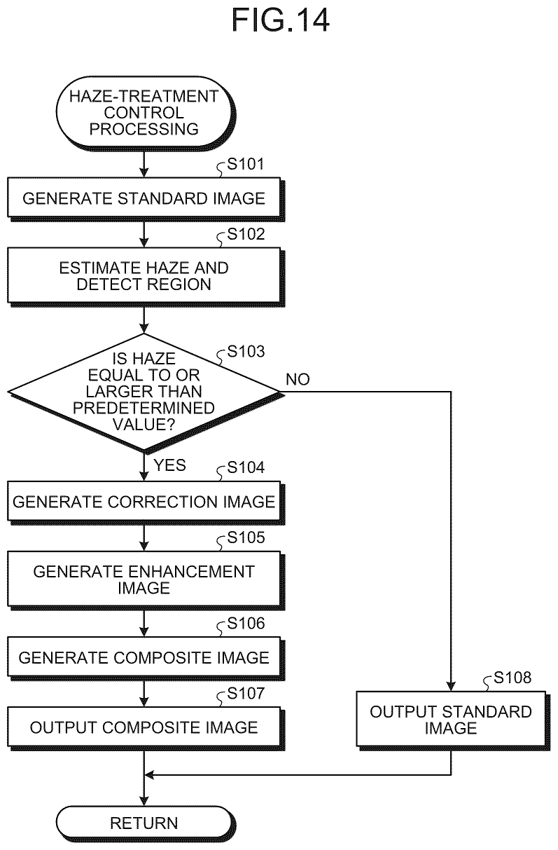

FIG. 14 is a flowchart illustrating a detailed overview of the haze-treatment control processing in FIG. 13.

As illustrated in FIG. 14, the image generating unit 222 generates the standard image (step S101). Specifically, the image generating unit 2222 generates the standard image (color image by visible light) based on image data input from the image-data input unit 2221.

Subsequently, the haze detecting unit 2223 estimates the haze in the field of view of the endoscope 201 based on the standard image generated by the image generating unit 2222, and the feature-amount detecting unit 2225 detects a region including at least a part of the treatment instrument 301, which is the energy treatment instrument, from the standard image (step S102). Specifically, the haze detecting unit 2223 estimates the haze component of the field of view of the endoscope 201 by an estimating method similar to that of the haze estimating unit 2226a described above. Furthermore, the feature-amount detecting unit 2225 detects a region including at least a part of the treatment instrument 301 by performing edge extraction to extract an edge component with respect to the standard image. The feature-amount detecting unit 2225 may, of course, perform brightness extraction processing to extract a brightness component with respect to the standard image, and detect a region having a brightness value equal to or larger than a predetermined brightness value as a region including at least a part of the treatment instrument 301. Moreover, the edge extraction processing may be performed by combining, for example, one or more of known filters, such as the Sobel filter, the Laplacian filter, and the Canny filter.

Thereafter, the haze determining unit 2224 determines whether the haze in the field of view of the endoscope 201 detected by the haze detecting unit 2223 is equal to or larger than a predetermined value (step S103). When it is determined that the haze component in the field of view of the endoscope 201 detected by the haze detecting unit 2223 is equal to or larger than the predetermined value by the haze determining unit 2224 (step S103: YES), the endoscope control device 202 shifts to step S104 described later. On the other hand, when it is determined that the haze component in the field of view of the endoscope 201 detected by the haze detecting unit 2223 is not equal to or larger than the predetermined value by the haze determining unit 2224 (step S103: NO), the endoscope control device 202 shifts to step S109 described later.

At step S104, the correction-image generating unit 2226 performs tone correction to remove or reduce the haze with respect to the standard image input from the image generating unit 2222 based on the detection result input from the haze detecting unit 2223, to generate the correction image. After step S104, the endoscope control device 202 shifts to step S105 described later.

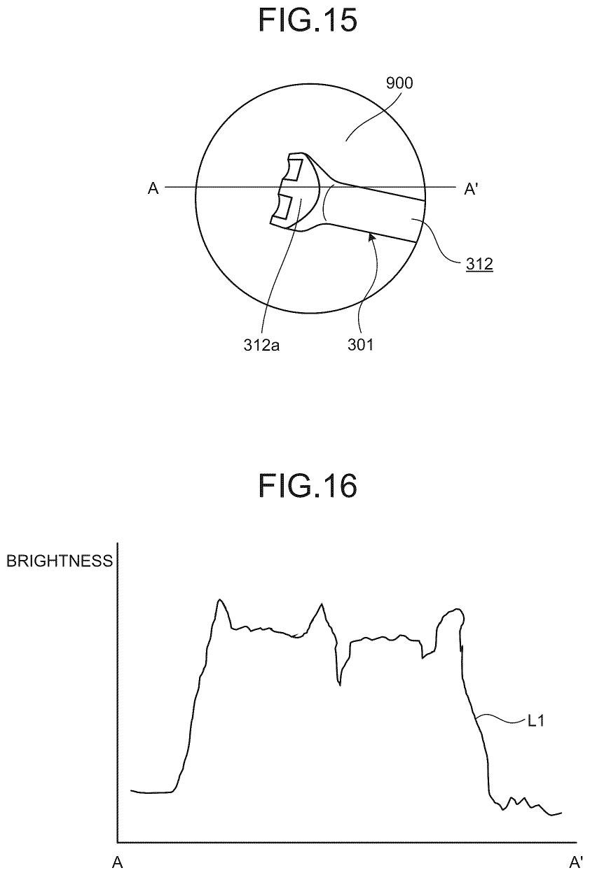

FIG. 15 is a diagram illustrating an example of a display image in a state in which a field of view of the endoscope 201 is in a good condition. FIG. 16 is a diagram illustrating a relationship between a position and a brightness on a line A-A′ in FIG. 15.

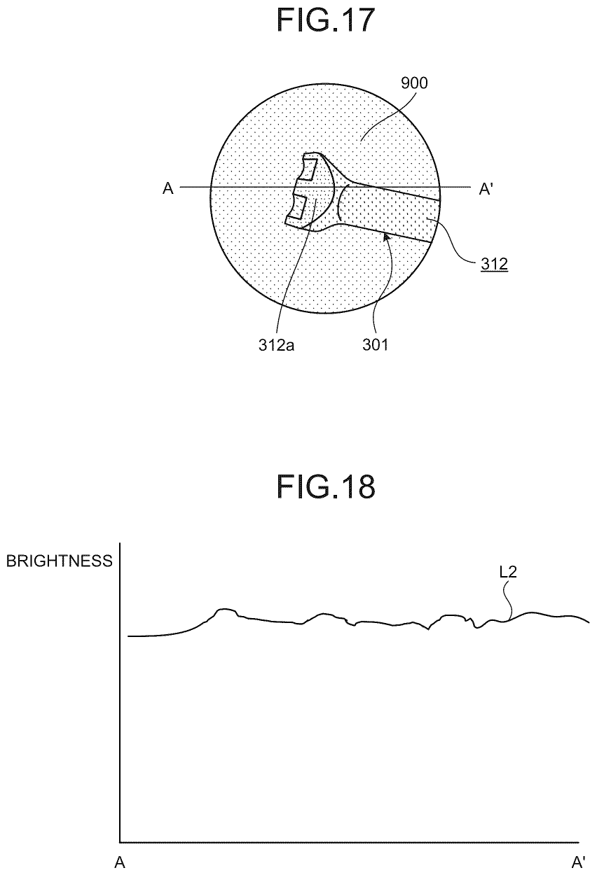

FIG. 17 is a diagram illustrating an example of a display image in a state in which a field of view of the endoscope 201 is in a poor condition.

FIG. 18 is a diagram illustrating a relationship between a position and a brightness on the line A-A′ in FIG. 16.

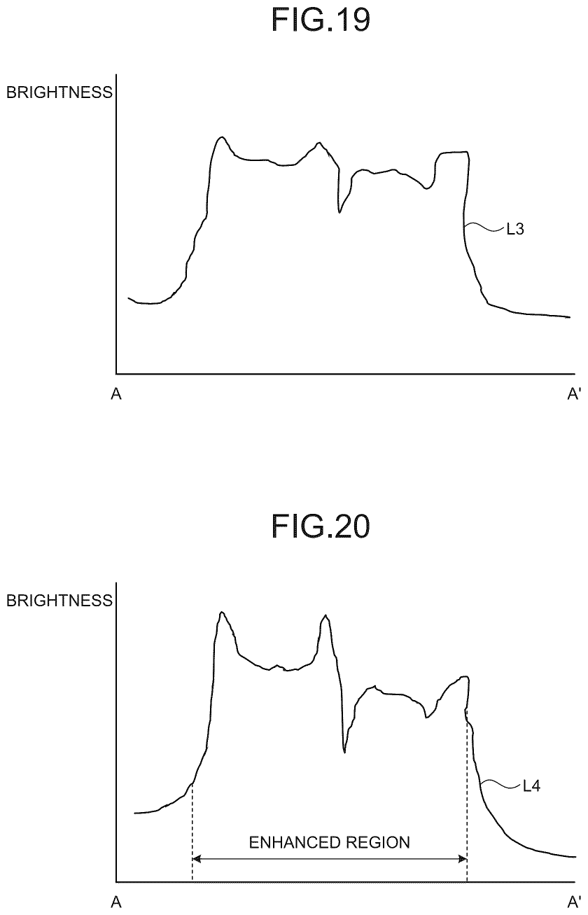

FIG. 19 is a diagram illustrating a relationship between a position and a brightness on the line A-A′ after the correction-image generating unit 2226 performs tone correction with respect to the display image in FIG. 17.

In FIG. 16, FIG. 18, and FIG. 19, a horizontal axis represents a position on the display image, and a vertical axis represents a brightness. Moreover, a curve L1 in FIG. 16 represents the relationship between the brightness and the position on the display image, and a curve L2 in FIG. 18 represents the relationship between the brightness and the position on the display image, and a curve L3 in FIG. 19 represents the relationship between the brightness and the position on the display image.

As indicated in FIG. 15 to FIG. 19, when the field of view of the endoscope 201 has changed from the good condition to the poor condition, the brightness increases (the curve L1 in FIG. 16 to the curve L2 in FIG. 18) even in a region other than the region in which the ultrasound probe 312 and the like are captured, as the field of view of the endoscope 201 becomes cloudy due to bone powder and the like (refer to a display image Q1 in FIG. 15 to a display image Q2 in FIG. 17). Therefore, the correction-image generating unit 2226 performs the tone correction to remove or reduce the haze with respect to the standard image input from the image generating unit 2222 based on the detection result input from the haze detecting unit 2223, to generate the correction image. In this case, as indicated by the curve L3 in FIG. 19, the correction-image generating unit 2226 generates the correction image such that the brightness of the region in which at least the ultrasound probe 312 and the treatment target site 100 are captured is high and the brightness in other regions is low.

Returning back to FIG. 14, explanation of step S105 and later will be continued.

At step S105, the enhancement-image generating unit 2227 generates the enhancement image by performing the edge enhancement processing with respect to the correction image generated by the correction-image generating unit 2226 based on the detection result detected by the feature-amount detecting unit 2225. After step S105, the endoscope control device 202 shifts to step S106.

FIG. 20 is a diagram illustrating a relationship between a position and a brightness of the enhancement image generated by the enhancement-image generating unit 2227.

In FIG. 20, a relationship between a position on the line A-A′ same as that in FIG. 15 and a brightness will be explained. Moreover, in FIG. 20, a horizontal axis represents a position on the enhancement image and a vertical axis represents a brightness. Furthermore, a curve LA in FIG. 20 represents the relationship between the brightness and the position on the enhancement image.

As indicated by the curve LA in FIG. 20, the enhancement-image generating unit 2227 performs the edge enhancement processing with respect to a region detected by the feature-amount detecting unit 2225 included in the correction image generated by the correction-image generating unit 2226, to generate an enhancement image in which the edge component is enhanced compared to other regions. Specifically, the enhancement-image generating unit 2227 performs the edge enhancement processing such that a contour (enhanced region) of the region in which at least the ultrasound probe 312 and the treatment target site 100 are captured is enhanced, to generate the enhancement image. The enhancement-image generating unit 2227 may generate an enhancement image in which a contour of a region in which the treatment target site 100 is captured is emphasized, for example, with a color specified by the operator, such as red and green, other than by the edge enhancement processing.

Returning back to FIG. 14, explanation of step S106 and later will be continued.

At step S106, the composite-image generating unit 2228 generates the composite image in which the standard image generated by the image generating unit 2222, and at least one of the correction image generated by the correction-image generating unit 2226 and the enhancement image generated by the enhancement-image generating unit 2227 are combined. After step S106, the endoscope control device 202 shifts to step S107 described later.

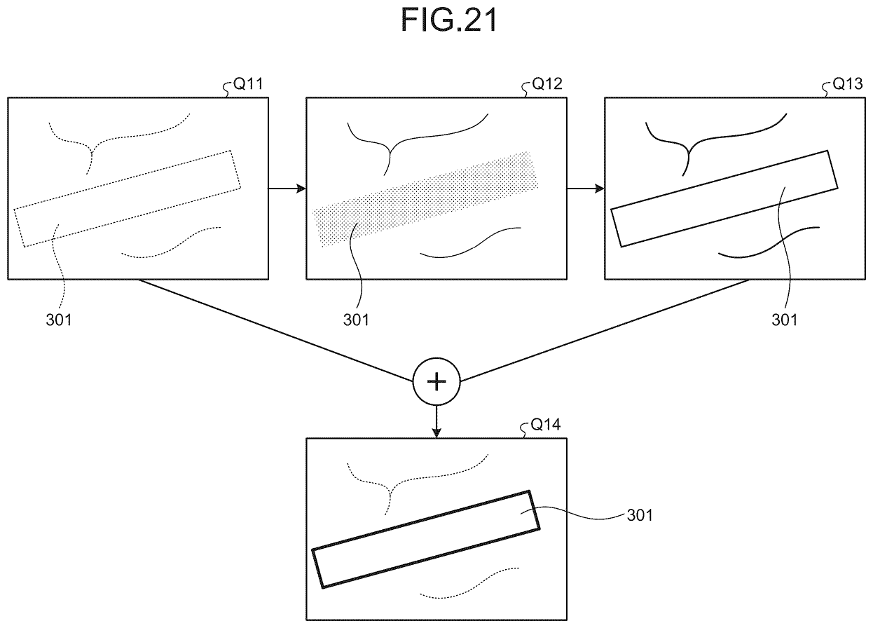

FIG. 21 is a diagram schematically illustrating a generation method of the composite image generated by a composite-image generating unit 2228.

As illustrated in FIG. 21, the composite-image generating unit 2228 generates a composite image Q14 by combining a standard image Q11, a correction image Q12, and an enhancement image Q13 at a predetermined ratio.

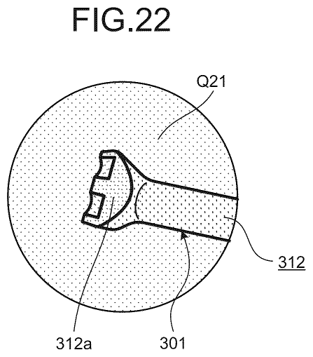

At step S107, the display-image generating unit 2229 generates a display image based on the composite image generated by the composite-image generating unit 2228, to output to the display device 203. FIG. 22 is a diagram illustrating an example of the display image displayed by the display device 203. As illustrated in FIG. 22, the operator can perform the cutting procedure with respect to the treatment target site 100 with the ultrasound probe 312 without interrupting because the contour of the treatment instrument 301 captured in a part of the display image Q21 is enhanced. After step S107, the endoscope control device 202 returns to the main routine of the cutting procedure in FIG. 13, and shifts to step S17.

At step S108, the display-image generating unit 2229 generates the display image based on the standard image generated by the image generating unit 2222, to output to the display device 203. After step S108, the endoscope control device 202 returns to the main routine of the cutting procedure in FIG. 13, and shifts to step S17.

According to the first embodiment explained above, because the display-image generating unit 2229 generates a display image based on the enhancement image input from the enhancement-image generating unit 2227, to output to the display device 203, even when the field of view in the endoscope 201 is deteriorated, a treatment of the treatment target site 100 can be continued because the position of the treatment instrument 301 can be grasped.

Moreover, according to the first embodiment, the composite-image generating unit 2228 generates a composite image in which the correction image generated by the correction-image generating unit 2226 and the enhancement image generated by the enhancement-image generating unit 2227 are combined, and the display-image generating unit 2229 generates a display image based on the composite image, to output to the display device 203. As a result, because the position of the treatment instrument 301 can be grasped even when the field of view of the endoscope 201 is deteriorated, the treatment on the treatment target site 100 can be continued.

Furthermore, according to the first embodiment, when it is determined that the haze is equal to or larger than the predetermined value by the haze determining unit 2224, the display-image generating unit 2229 generates a display image based on the enhancement image input from the enhancement-image generating unit 2227, to output to the display device 203. On the other hand, when it is determined that the haze is not equal to or larger than the predetermined value by the haze determining unit 2224, the display-image generating unit 2229 outputs a display image based on the standard image generated by the image generating unit 2222. This enables the operator to perform treatment while viewing the display image, which is the standard image, when the field of view of the endoscope 201 is in a good condition, and to perform the treatment while viewing the display image in which the treatment instrument 301 is enhanced when the field of view of the endoscope 201 is in a poor condition.

In the first embodiment, the composite-image generating unit 2228 may generate a composite image in which the standard image generated by the image generating unit 2222 and the enhancement image generated by the enhancement-image generating unit 2227 are combined, and the display-image generating unit 2229 may generate a display image based on the composite image, to output to the display device 203.

### Second Embodiment

Next, a second embodiment will be explained. In the first embodiment described above, the composite image in which the standard image or the correction image and the enhancement image are combined is generated, but in the second embodiment, a composite image in which a position of a treatment instrument of a standard image before occurrence of haze and a position of the treatment instrument captured in a correction image are combined is generated, to be output to the display device. Specifically, a configuration of an image processing unit according to the second embodiment and haze-treatment control processing are different. Therefore, in the following, after the configuration of the image processing unit according to the second embodiment is explained, the haze-treatment control processing performed by the endoscope control device 202 according to the second embodiment will be explained.

### Configuration of Image Processing Unit

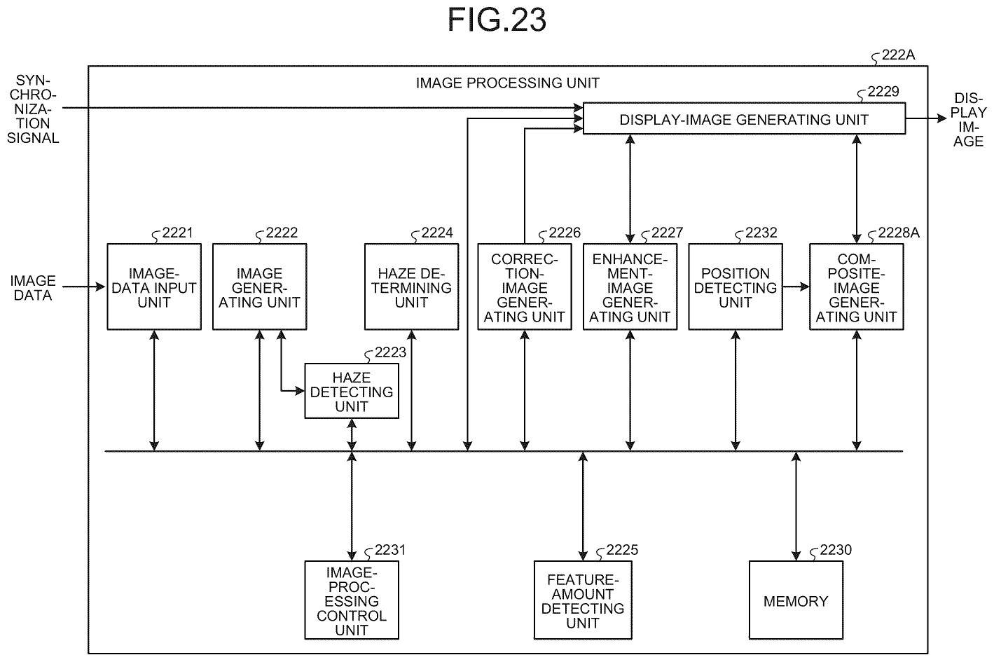

FIG. 23 is a block diagram illustrating a functional configuration of the image processing unit according to the second embodiment.

An image processing unit 222A illustrated in FIG. 23 further includes a position detecting unit 2232 in addition to the configuration of the image processing unit 2222 according to the first embodiment described above. Furthermore, the image processing unit 222A includes a composite-image generating unit 2228A in place of the composite-image generating unit 2228 according to the first embodiment described above.

The position detecting unit 2232 detects a part of the treatment instrument that is captured in the standard image generated by the image generating unit 2222. Specifically, the position detecting unit 2232 detects a feature amount of each pixel in the standard image generated by the image generating unit 2222 or the correction image generated by the correction-image generating unit 2226, and the enhancement image generated by the enhancement-image generating unit 2227, and detects a matching portion based on this feature amount. The feature amount includes, for example, an edge component, a brightness component, and the like. The position detecting unit 2232 may detect the treatment instrument 301 that is captured in the standard image by using a technique of publicly-known pattern matching processing, and may output this detection result to the composite-image generating unit 2228A.

The composite-image generating unit 2228A generates a composite image in which the standard image before a haze is detected therein stored in the memory 2230, and the correction image generated by the correction-image generating unit 2226 are combined based on the detection result detected by the position detecting unit 2232.

### Details of Haze-Treatment Control Processing

Next, details of the haze-treatment control processing performed by the endoscope control device 202 according to the second embodiment will be explained.

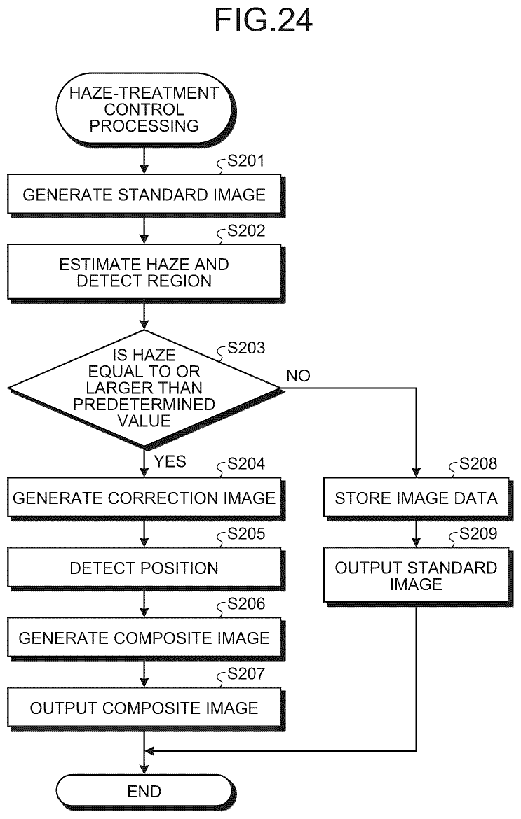

FIG. 24 is a flowchart illustrating a detailed overview of the haze-treatment control processing performed according to an instruction by the CPU 227 of the endoscope control device 202 according to the second embodiment to the respective components through a bus.

Step S201 to step S204 correspond to step S101 to step S104 in FIG. 14, respectively.

The position detecting unit 2232 detects the position of a matching portion in which the standard image generated by the image generating unit 2222 or the correction image generated by the correction-image generating unit 2226, and the enhancement image generated by the enhancement-image generating unit 2227 match (step S205).

Subsequently, the composite-image generating unit 2228A performs composition processing to generate a composite image in which the standard image before a haze is detected therein stored in the memory 2230 and the correction image generated by the correction-image generating unit 2226 are combined, based on the detection result detected by the position detecting unit 2232 (step S206). The standard image before a haze is detected therein stored in the memory 2230 is used as the enhancement image in the second embodiment.

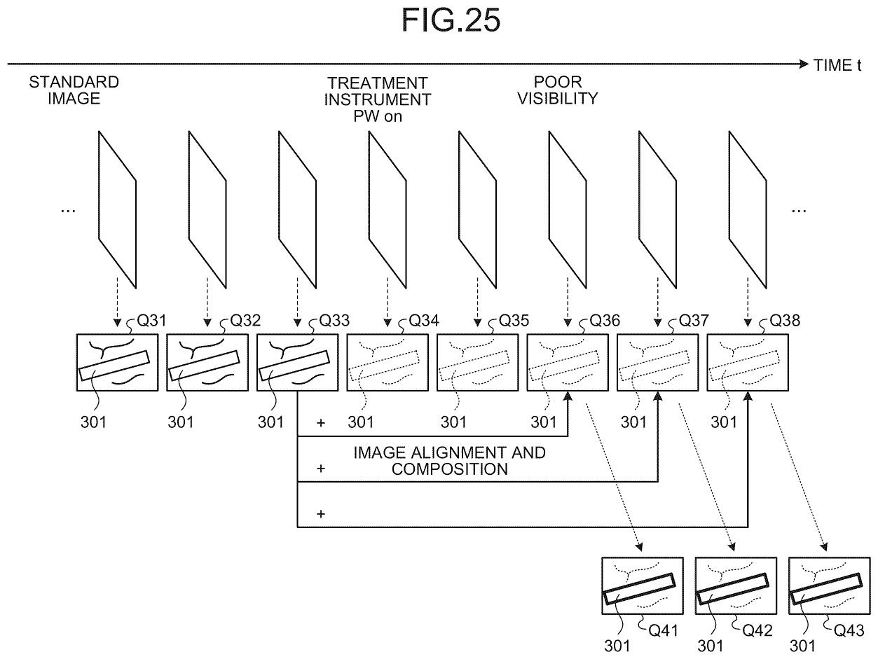

FIG. 25 is a diagram schematically illustrating a generation method of a composite image that is generated by the composite-image generating unit 2228A.

As illustrated in FIG. 25, the composite-image generating unit 2228A sequentially generates, when the treatment instrument 301 is activated (PW on) and temporally continuous standard images (a standard image Q31 to a standard image Q33), which are the field of view of the endoscope 201, gradually become cloudy to be a standard image with a poor visibility (a standard image Q34 to a standard image Q38), composite images (composite images Q41 to Q43) by combining the standard image before a haze is detected therein stored in the memory 2230 (for example, the standard image Q33) and the correction image generated by the correction-image generating unit 2226. Specifically, the composite-image generating unit 2228A sequentially generates the composite images (the composite images Q41 to Q43) by combining the standard image (for example, the standard image Q33) at, at least one of a time of start of treatment, a time soon after start of treatment and a time soon before start of treatment by the treatment instrument 301 on the treatment target site 100, and the correction image generated by the correction-image generating unit 2226, based on the driving signal of the treatment instrument 301, the standard image being before a haze is detected and stored in the memory 2230. Thus, the operator can perform the cutting procedure on the treatment target site 100 by the treatment instrument 301 without interrupting while viewing a smooth display image displayed on the display device 203 even when the field of view of the endoscope 201 turns into a poor condition because the contour of the treatment instrument 301 is enhanced.

After step S206, the display-image generating unit 2229 generates a display image based on the composite image generated by the composite-image generating unit 2228, to output to the display device 203 (step S207). After step S206, the endoscope control device 202 returns to the main routine of the cutting procedure in FIG. 13, and shifts to step S17.

At step S208, the image-processing control unit 2231 stores the image data, which is the standard image generated by the image generating unit 2222, in the memory 2230.

Subsequently, the display-image generating unit 2229 generates a display image based on the standard image generated by the image generating unit 2222, to output to the display device 203 (step S209). After step S209, the endoscope control device 202 returns to the main routine of the cutting procedure in FIG. 13, and shifts to step S17.

According to the second embodiment explained above, an effect similar to that of the first embodiment described above can be produced, and even when the field of view of the endoscope 201 is deteriorated, a treatment of the treatment target site 100 by using the treatment instrument 301 can be continued.

### Third Embodiment

Next, a third embodiment will be explained. In the third embodiment, a mark is arranged near a distal end of the treatment instrument, this mark arranged near the distal end of the treatment instrument is detected, and an enhancement image is generated based on this detection result. Specifically, in the third embodiment, configurations of the image processing unit and the treatment instrument are different. In the following, the configurations of the image processing unit and the treatment instrument according to the third embodiment will be explained. Identical reference symbols are assigned to identical components to those of the treatment system 1 according to the first embodiment described above, and detailed explanation thereof will be omitted.

### Functional Configuration of Image Processing Unit

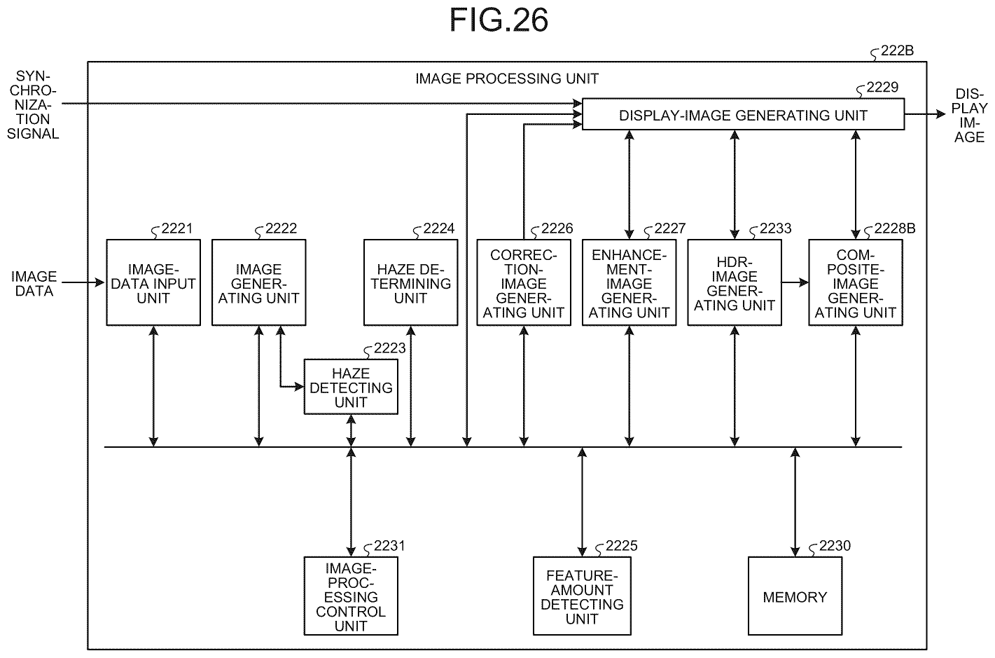

FIG. 26 is a block diagram illustrating a functional configuration of the image processing unit according to the third embodiment.

An image processing unit 222B illustrated in FIG. 26 includes a high dynamic range (HDR)-image generating unit 2233 in addition to the configuration of the image processing unit 222 according to the first embodiment described above. Furthermore, a composite-image generating unit 2228B is provided in place of the composite-image generating unit 2228 according to the first embodiment described above.

The HDR-image generating unit 2233 generates an HDR image using two images of different exposure, and outputs this HDR image to the display-image generating unit 2229. Specifically, the HDR image is generated using two standard images of different exposure.

The enhancement-image generating unit 2227 detects an image region corresponding to the mark near the distal end of the treatment instrument from the HDR image, and generates an enhancement image obtained by performing the edge enhancement processing according to a degree of edge visibility in image data.

The composite-image generating unit 2228B generates a composite image in which the HDR image generated by the HDR-image generating unit 2233 and at least either one of the correction image generated by the correction-image generating unit 2226 and the enhancement image generated by the enhancement-image generating unit 2227 are combined.

### Configuration of Treatment Instrument

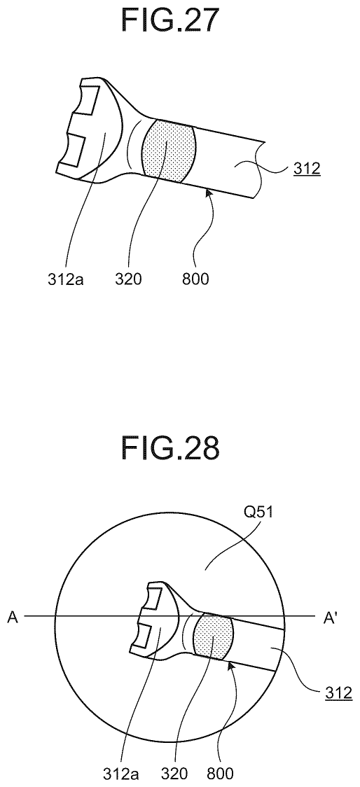

FIG. 27 is a schematic diagram illustrating a schematic configuration of a part of the treatment instrument according to the third embodiment.

A treatment instrument 301B illustrated in FIG. 27 includes a retroreflective portion 320 that functions as an indicator arranged on a proximal end side relative to the ultrasound transducer 312a along the longitudinal direction, in addition to the configuration of the treatment instrument 301 according to the first embodiment described above. The retroreflective portion 320 reflects illumination light emitted from the endoscope 201. The retroreflective portion 320 is constituted of a retroreflective material, and the like.

The image processing unit 222B thus configured performs the haze-treatment control processing similar to that in the first and the second embodiments described above. In this case, the standard image corresponding to the field of view of the endoscope 201 is to exceed a dynamic range of the imaging device 2241 because of an overexposed areas caused by the reflected light from the retroreflective portion 320 in the treatment instrument 301B. Therefore, the HDR-image generating unit 2233 generates an HDR image using two images of different exposure, and thereby prevents overexposure of an image even when the retroreflective portion 320 is arranged in the treatment instrument 301.

FIG. 28 is a diagram illustrating an example of a display image in which a part of the treatment instrument 301B captured in the field of view of the endoscope 201.

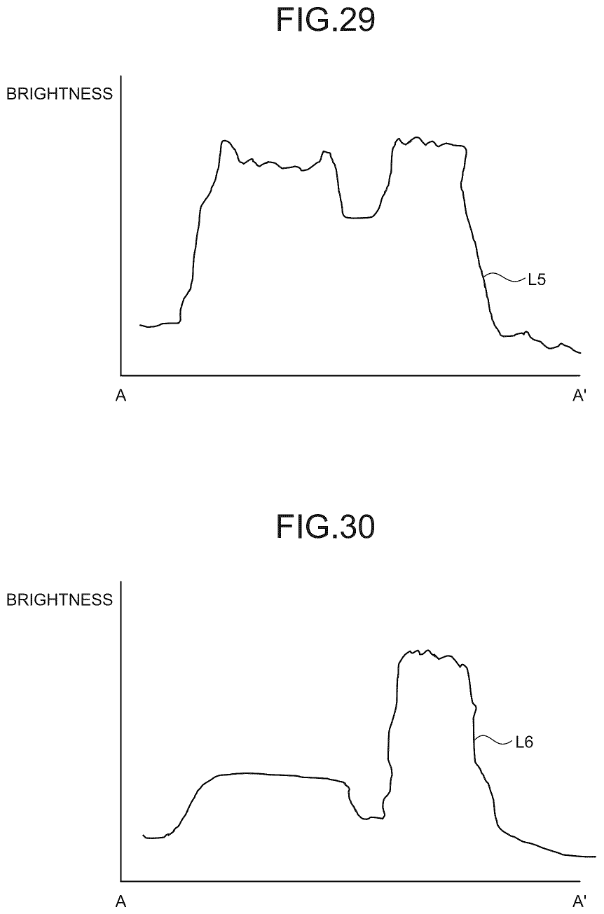

FIG. 29 is a diagram illustrating a relationship between a position on a line A-A′ in FIG. 28 and a brightness.

FIG. 30 is a diagram illustrating a relationship between a position on the same line as the line A-A′ in FIG. 28 and a brightness in the HDR image generated by the HDR-image generating unit 2233.

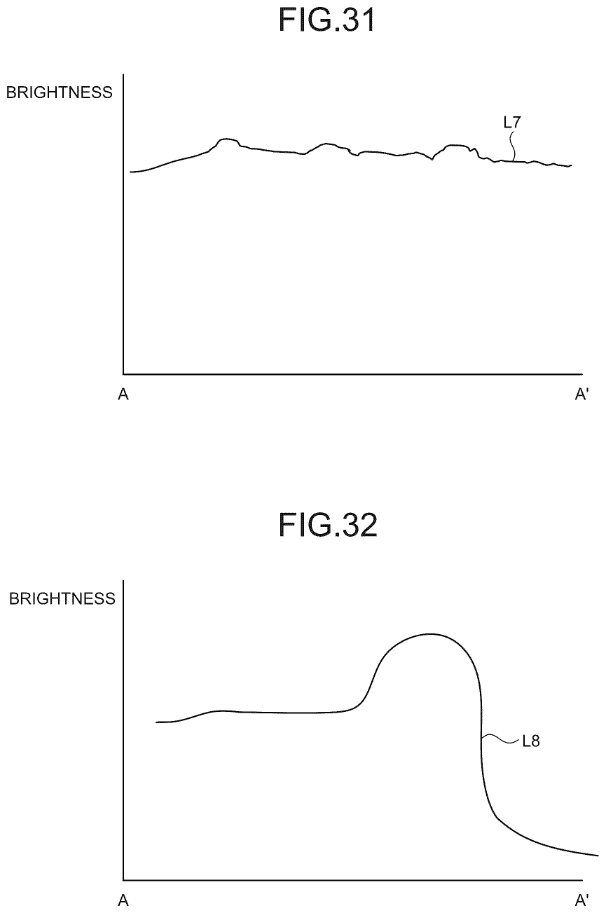

FIG. 31 is a diagram illustrating a relationship between a position on the same lie as the line A-A′ in FIG. 28 and a brightness in the display image when the field of view of the endoscope 201 is in a poor condition.

FIG. 32 is a diagram illustrating a relationship between a position on the same line as the line A-A′ in FIG. 28 and a brightness in the composite image generated by the composite-image generating unit 2228A.

In FIG. 29 to FIG. 32, a horizontal axis represents a position in an image, and a vertical axis represents a brightness.

Moreover, a curve L5 to a curve L8 in FIG. 29 to FIG. 32 indicates a relationship between a brightness and a position in each image.

As indicated by the curve L5 in FIG. 29 and the curve L6 in FIG. 30, the standard image corresponding to the field of view of the endoscope 201 is to exceed a dynamic range of the imaging device 2241 because of an overexposed areas caused by the reflected light from the retroreflective portion 320 in the treatment instrument 301B. Furthermore, as indicated by the curve L7 in FIG. 31, the standard image when the field of view of the endoscope 201 is in a poor condition is to exceed a dynamic range of the imaging device 2241 due to the turbidity of the irrigation fluid.

Therefore, as indicated by the curve L8 in FIG. 32, the HDR-image generating unit 2233 generates an HDR image using two correction images of different exposure and the standard image, and thereby prevents overexposure in an image even when the retroreflective portion 320 is arranged in the treatment instrument 301B.

According to the third embodiment explained above, a similar effect to that of the first embodiment described above can be produced, and even when the field of view of the endoscope 201 is deteriorated, a treatment of the treatment target site 100 by using the treatment instrument 301B can be continued.

### Fourth Embodiment

Next, a fourth embodiment will be explained. In the fourth embodiment, movement of a treatment instrument is detected, and this detection result is displayed. Specifically, in the fourth embodiment, configurations of the image processing unit and the treatment instrument are different. In the following, configurations of the image processing unit and the treatment instrument according to the fourth embodiment will be explained. Identical reference symbols are assigned to identical components to those of the treatment system 1 according to the first embodiment described above, and detailed explanation thereof will be omitted.

### Functional Configuration of Image Processing Unit

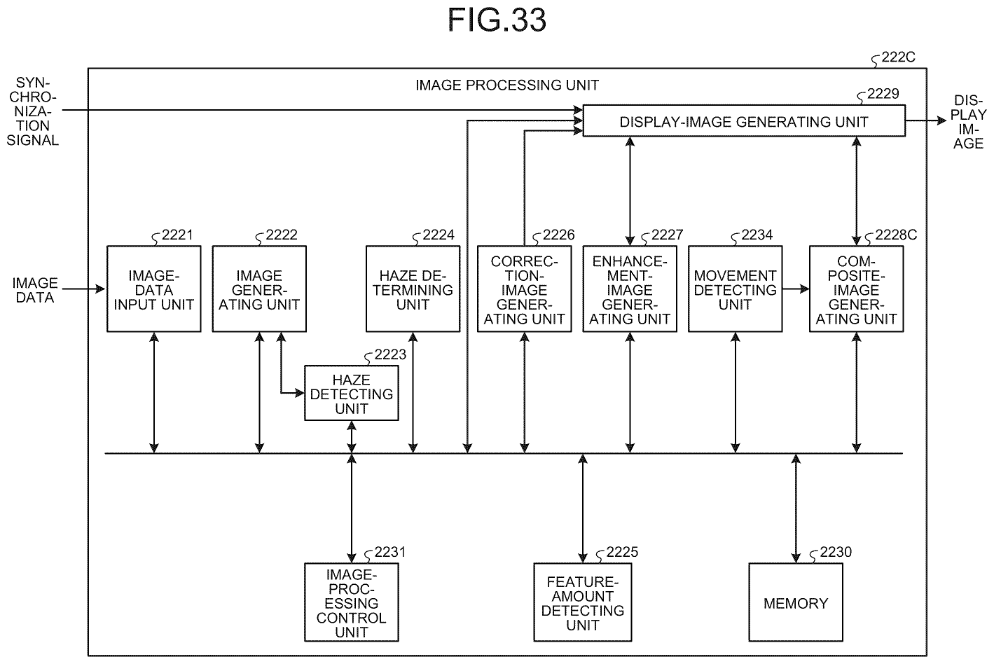

FIG. 33 is a block diagram illustrating a functional configuration of the image processing device according to the fourth embodiment.

An image processing unit 222C illustrated in FIG. 33 further includes a movement detecting unit 2234 in addition to the configuration of the image processing unit 222 according to the first embodiment described above. Furthermore, the image processing unit 222C includes a composite-image generating unit 2228C in place of the composite-image generating unit 2228.

The movement detecting unit 2234 detects a movement amount of the treatment instrument based on a scale portion arranged in the treatment instrument that is captured in the standard image or the correction image described later, and outputs this detection result to the composite-image generating unit 2228C. Specifically, the movement detecting unit 2234 detects the movement amount of the treatment instrument by detecting a scale of the scale portion, for example, by performing publicly-known pattern matching on the scale portion captured in the temporally continuous standard images or the correction image. The movement amount is an amount of movement for which the distal end portion of the ultrasound transducer 312a moves, for example, when the treatment instrument performs a treatment on the treatment target site 100.

The composite-image generating unit 2228C generates a composite image in which the correction image or the enhancement image is combined with information relating to the movement amount of the treatment instrument based on the detection result of the movement detecting unit 2234.

### Configuration of Treatment Instrument

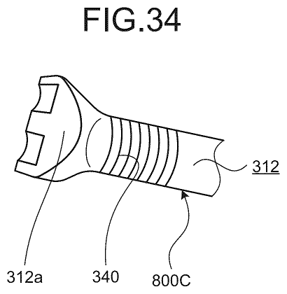

FIG. 34 is a schematic diagram illustrating a schematic configuration of a part of the treatment instrument according to the fourth embodiment.

A treatment instrument 301C illustrated in FIG. 34 includes an indicator 340 that is formed at predetermined intervals on the proximal end side relative to the ultrasound transducer 312a along the longitudinal direction in addition to the configuration of the treatment instrument 301 described above.

The image processing unit 222C thus configured performs the haze-treatment control processing similar to that of the first and the second embodiments described above. In this case, the composite-image generating unit 2228C combines the correction image or the enhancement image with the information relating to the movement amount of the treatment instrument 301C based on the detection result of the movement detecting unit 2234. The display-image generating unit 2229 generates a display image using the composite image generated by the composite-image generating unit 2228C, and outputs this display image to the display device 203. Thus, the operator can perform a treatment by the treatment instrument 301C without interrupting because the movement amount of the treatment instrument 301C is displayed and the operator can grasp the movement amount of the treatment instrument C, even when the field of view of the endoscope 201 is deteriorated as a result of the treatment by the treatment instrument 301C.

According to the fourth embodiment explained above, an effect similar to that of the first embodiment described above is produced, and even when the field of view of the endoscope 201 is deteriorated, a treatment of the treatment target site 100 by using the treatment instrument 301C can be continued.

### Modification According to First to Fourth Embodiments

In the first to the fourth embodiments described above, the image to be output to the display device 203 by the display-image generating unit 2229 is switched according to the mode set to the endoscope control device 202, but it is not limited thereto. For example, the image to be output to the display device 203 by the display-image generating unit 2229 may be switched based on the driving signal and the synchronization signal (VT) input from the treatment-instrument control device 302. Specifically, the display-image generating unit 2229 outputs either one of the correction image, the enhancement image, and the composite image to the display device 203 when either one of the driving signal to drive the treatment instrument 301 and the synchronization signal (VT) is input from the treatment-instrument control device 302.

This enables to switch contents of the display image to be displayed on the display device 203 without switching the mode of the endoscope control device 202 each time and, therefore, the operator can perform the cutting procedure with respect to the treatment target site 100 with the ultrasound probe 312 without complicated operations.

Furthermore, because the display-image generating unit 2229 switches the type of the image to be output to the display device 203 according to the synchronization signal, the type of the image to be displayed on the display device 203 is switched smoothly. Therefore, it is possible to prevent discomfort for the operator and reduce burden on the operator.

### Other Embodiments

Moreover, in the first to the fourth embodiments, it has been explained about the treatment for turbidity caused by bone powder and the like in fluid, such as the irrigation fluid, but it is not limited to that in fluid, and it can also be applied in air. In the first to the third embodiments, it can also be applied to the deterioration of visibility in the field of view of an endoscope caused by cutting debris, fat mist, and the like resulting from a procedure in air in a joint area.

Furthermore, in the first to the fourth embodiments of the present disclosure, it has been explained about the treatment of a knee joint, but it can be applied to other parts (spine or the like), not limited to the knee joint.

Moreover, the first to the fourth embodiments of the present disclosure can be applied to turbidity due to factors other than bone powder. For example, it can also be applied to debris, such as soft tissue, synovium, and fat, and other noise (cavitation, such as bubbles). For example, in the first to the third embodiments, as a factor of deterioration of the field of view caused by a treatment by the treatment instrument 301, it can also be applied to turbidity or visibility degradation caused by cutting debris of soft tissue such as cartilage, synovium, and fat as tissue fragments.

Furthermore, the first to the fourth embodiments of the present disclosure can be applied to the deterioration of visibility caused by fine bubbles resulting from factors, such as cavitation associated with ultrasound vibrations of the treatment instrument 301 in a procedure in liquid using the treatment instrument 301.

Moreover, the first to the fourth embodiments of the present disclosure can be applied even when the field of view of the endoscope 201 is obstructed by a relatively large tissue fragment. In this case, the endoscope control device 202 may be configured to determine whether the field of view of the endoscope 201 is obstructed by an obstruction based on the standard image, and to perform image processing to remove the obstruction by using a publicly-known technique when it is determined to be obstructed by an obstruction. In this case, the endoscope control device 202 may perform the image processing to the extent that it does not affect the processing, using the size of a treatment area by the treatment instrument 301, a duration for which the treatment target site 100 is occluded, and the like.

Furthermore, by combining the multiple components disclosed in the treatment system according to the first to the fourth embodiments of the present disclosure, various embodiments can be formed. For example, from the entire components described in the treatment system according to the first to the third embodiment of the present disclosure described above, some of the components may be removed. Furthermore, the components explained in the treatment system according to the first to the third embodiments of the present disclosure described above may be combined as appropriate.

Moreover, in the treatment system according to the first to the fourth embodiments of the present disclosure, “unit” that has been used in the above description can be replaced with “means”, “circuit”, or the like. For example, the control unit may be referred to as control means or control circuit.

Furthermore, a program that is executed by the treatment system according to the first to the fourth embodiments of the present disclosure is stored in a computer-readable storage medium, such as a compact disk read-only memory (CD-ROM), a flexible disk (FD), a compact disk rewritable (CD-R), a digital versatile disk (DVD), a USB medium, and a flash memory, in a form of file data in an installable format or in an executable format, to be provided.

Moreover, a program executed by the treatment system according to the first to the fourth embodiments of the present disclosure may be stored in a computer that is connected through a network, such as the Internet, to be provided by being downloaded through the network.

Furthermore, in the explanation of flowcharts in the present specification, expressions such as “first”, “thereafter”, “subsequently”, and the like are used to indicate the sequence of processing between steps. However, the order of processing to implement the present disclosure is not uniquely determined by these expressions. That is, the sequence of processing in the flowcharts described in the present specification can be changed within a range not causing contradictions. Moreover, not limited to a program constituted of simple branching processing as described, more judgment criteria may be comprehensively determined to branch.

According to the present disclosure, an effect is produced that a procedure on a treatment site can be continued even when the field of view of an endoscope is deteriorated.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising a processor comprising hardware, the processor being configured to:
acquire image data partially including a first region in which a living body is treated with at least an energy treatment instrument;
detect a second region including at least a part of the energy treatment instrument from an image corresponding to the image data;
generate enhancement image data in which the second region is enhanced compared to regions other than the second region, based on the image data and a detection result of the second region;
perform tone correction on the image corresponding to the image data to generate correction image data; and
generate first composite image data in which the correction image data and the enhancement image data are combined.

2. The image processing device according to claim 1, wherein
the processor is further configured to generate a display image based on the first composite image data.

3. The image processing device according to claim 1, wherein
the processor is further configured to detect a marking portion arranged in the energy treatment instrument.

4. The image processing device according to claim 3, wherein
the marking portion is a retroreflective material.

5. The image processing device according to claim 3, wherein
the marking portion is a scale arranged at predetermined intervals.

6. The image processing device according to claim 1, wherein
the processor is further configured to:
detect a haze in each of the image data sequentially acquired;
determine whether the haze is equal to or larger than a predetermined value, for each detection of the haze;
generate a display image based on the enhancement image data when it is determined that the haze is equal to or larger than the predetermined value; and
generate a display image based on the image data when it is determined that the haze is not equal to or larger than the predetermined value.

7. The image processing device according to claim 1, wherein
the processor is further configured to detect the second region by detecting either one of an edge component, a brightness, and a movement amount included in the image.

8. The image processing device according to claim 1, wherein
the processor is further configured to:
generate second composite image data in which the image data and the enhancement image data are combined;
acquire the image data at, at least one of a time of start of treatment and a time soon after start of treatment with the energy treatment instrument on a treatment target site based on a driving signal of the energy treatment instrument; and
generate a display image based on the second composite image data.

9. The image processing device according to claim 8, wherein
the processor is further configured to:
detect a haze in each of the image data sequentially acquired;
determine whether the haze is equal to or larger than a predetermined value, for each detection of the haze; and
generate the second composite image data by combining the image data acquired just before the haze is determined to be equal to or larger than the predetermined value with the enhancement image data.

10. The image processing device according to claim 1, wherein
the processor is further configured to perform at least one of edge enhancement processing and tone correction processing with respect to the region to generate the enhancement image data.

11. The image processing device according to claim 1, wherein
the processor is further configured to output at least one of a display image based on the image data and a display image based on the enhancement image data, to a display.

12. An energy treatment instrument that can be inserted into a subject and is capable of treating a treatment target site, the energy treatment instrument be configured to be captured by an endoscope inserted into the subject, the energy treatment instrument comprising
an indicator that is arranged at a distal end of the energy treatment instrument, the indicator being configured to be detected by enhancing a region included in a part of the energy treatment instrument in an image corresponding to image data captured by the endoscope compared to other regions in an edge component and a brightness.

13. A treatment system comprising:
an energy treatment instrument that can be inserted into a subject, and that is capable of treating a treatment target site;
an endoscope that can be inserted into the subject, and that is capable of generating image data by capturing a first region in which a living body is treated with at least the energy treatment instrument; and
an image processing device configured to perform image processing with respect to the image data to output to a display,
the image processing device comprising a processor comprising hardware, the processor being configured to:
acquire the image data;
detect a second region including at least a part of the energy treatment instrument from an image corresponding to the image data;
generate enhancement image data in which the second region is enhanced compared to regions other than the second region, based on the image data and a detection result of the second region;
perform tone correction on the image corresponding to the image data to generate correction image data; and
generate composite image data in which the correction image data and the enhancement image data are combined.

14. An image processing method that is performed by an image processing device comprising a processor comprising hardware, the method comprising:
acquiring image data partially including a first region in which a living body is treated with at least an energy treatment instrument;
detecting a second region including at least a part of the energy treatment instrument from an image corresponding to the image data;
generating enhancement image data in which the second region is enhanced compared to regions other than the second region, based on the image data and a detection result of the second region;
performing tone correction on the image corresponding to the image data to generate correction image data; and
generating first composite image data in which the correction image data and the enhancement image data are combined.
